(19) United States
(12) Patent Application Publication
(10) Pub. No.: US 2007/0264283 A1
(43) Pub. Date: Nov. 15, 2007

(54) VACCINE

(75) Inventors: Gerald Wayne Gough, Stevenage (GB); Christopher Michael Roberts, Stevenage (GB)

Correspondence Address: GLAXOSMITHKLINE;Corporate Intellectual Property - UW2220, P.O. Box 1539, King of Prussia, PA, 19406-0939, US

(73) Assignee: Glaxo Group Limited

(21) Appl. No.: 11/760,127

(22) Filed: Jun. 8, 2007

Related U.S. Application Data
(63) Continuation of application No. 10/529,931, filed on Jan. 5, 2006.

(30) Foreign Application Priority Data
Oct. 3, 2002 (GB) 0222953.2

(51) Int. Cl. A61K 39/12; A61K 31/7088; A61P 15/00; A61P 31/22; A61P 35/00; C07H 21/04; C12N 15/63

(52) U.S. Cl. 424/204.1; 435/320.1; 514/44; 536/23.72

(57) ABSTRACT

The present invention relates to methods and compositions useful in the treatment and prevention of human papilloma virus infections. In particular the invention relates to nucleic acid molecules encoding E1 and/or E2 and vectors suitable for DNA vaccine delivery, and pharmaceutical compositions containing them. Methods for manufacturing said molecules, vectors and composition are also contemplated.

### CROSS REFERENCE TO PRIOR APPLICATION

This is a continuation of application Ser. No. 10/529,931, filed 5 Jan. 2006, which is a 371 application of PCT/EP2003/011158 filed 1 Oct. 2003.

The present invention relates to methods and compositions useful in the treatment and prevention of human papilloma virus infections. In particular the invention relates to nucleic acid molecules typically encoding a polyprotein based on Early antigens from different HPV strains, and vectors suitable for DNA vaccine delivery, and pharmaceutical compositions containing them. Methods for manufacturing said molecules, vectors and composition are also contemplated, as are their use in medicine.

### BACKGROUND TO THE INVENTION

The papillomavirus virus is highly tissue and species specific. It infects basal epithelial cells and replicates and completes its full life cycle within the cell nucleus. Viral gene expression is tightly linked to epithelial cell differentiation and capsid assembly and maturation only occurs in fully differentiated epithelial cells in the upper epithelial cell layers.

The infecting human papillomavirus genotypes present in genital warts are known to be either genotype 6b or genotype 11. The majority (90%) of genital warts are infected with HPV6b, whilst approximately 10% are infected with HPV-11. The primary infecting genotypes present in infections relating to cervical carcinoma are HPV16 and 18.

Human genital warts may develop at the site of infection and they may become chronic, persisting for extended periods of time or, alternatively they may regress spontaneously resolving completely without scarring. The factors that trigger this regression are undefined but it is postulated that cellular response may be involved in the disease resolution process.

Papillomaviruses are not naturally very immunogenic and during the course of natural infection antibodies may only occur very late (during or after resolution), and in a fraction of patients whilst some patients may resolve disease without developing detectable antibody at all.

Vaccination using papillomavirus early antigens has been widely studied in several different animal model systems. However there are only a few reports studying therapeutic immunisation.

For example, cattle immunised therapeutically with a cocktail of proteins comprising bovine papillomavirus (BPV) proteins E1, E2, E4 and E7 showed a reduced papilloma disease burden in a proportion of animals compared to controls.

Papilloma virus infections have been observed in a variety of species, including sheep, dogs, rabbits, monkeys, cattle and humans. Human papilloma viruses (HPV) have been classified into more than 80 types [Epidemiology and Biology of Cervical Cancer Seminars in Surgical Oncology 1999 16:203-211. Wolfgang M J, Schoell M D, Janicek M F and Mirhashemi R.], some of which are further divided into sub-types (e.g. type 6a and 6b), based on the extent of DNA sequence homology. Papilloma viruses generally infect epithelia, but the different HPV types cause distinct diseases. For example, types 1-4, 7, 10 and 26-29 cause benign warts, types 16, 18, 31, 33, 35, 39, 45, 51, 52, 56, 58, 59, and 68 are associated with cervical cancers and types 6 and 11 are implicated in genital warts (non-malignant condylomata of the genital tract).

HPV has proven difficult to grow in tissue culture, so there is no traditional live or attenuated viral vaccine. Development of an HPV vaccine has also been slowed by the lack of a suitable animal model in which the human virus can be studied. This is because the viruses are highly species specific, so it is very difficult to infect an animal with a papilloma virus from a host of a different species, as would be required for safety testing before a vaccine was first tried in humans.

Papilloma viruses have a DNA genome which encodes “early” and “late” genes designated E1 to E7, L1 and L2. The early gene sequences have been shown to have functions relating to viral DNA replication and transcription, evasion of host immunity, and alteration of the normal host cell cycle and other processes. For example the E1 protein is an ATP-dependent DNA helicase and is involved in initiation of the viral DNA replication process whilst E2 is a regulatory protein controlling both viral gene expression and DNA replication. Through its ability to bind to both E1 and the viral origin of replication, E2 brings about a local concentration of E1 at the origin, thus stimulating the initiation of viral DNA replication. The E4 protein appears to have a number of poorly defined functions but amongst these may be binding to the host cell cytoskeleton, whilst E5 appears to delay acidification of endosomes resulting in increased expression of EGF receptor at the cell surface and both E6 and E7 are known to bind cell proteins p53 and pRB respectively. The E6 and E7 proteins form HPV types associated with cervical cancer are known oncogenes. L1 and L2 encode the two viral structural (capsid) proteins.

Historically, vaccines have been seen as a way to prevent infection by a pathogen, priming the immune system to recognise the pathogen and neutralise it should an infection occur. The vaccine includes one or more antigens from the pathogen, commonly the entire organism, either killed or in a weakened (attenuated) form, or selected antigenic peptides from the organism. When the immune system is exposed to the antigen(s), cells are generated which retain an immunological “memory” of it for the lifetime of the individual. Subsequent exposure to the same antigen (e.g. upon infection by the pathogen) stimulates a specific immune response which results in elimination or inactivation of the infectious agent.

There are two arms to the immune response: a humoral (antibody) response and a cell-mediated response. Protein antigens derived from pathogens that replicate intracellularly (viruses and some bacteria) are processed within the infected host cell releasing short peptides which are subsequently displayed on the infected cell surface in association with class I major histocompatability (MHC I) molecules. When this associated complex of MHC I and peptide is contacted by antigen-specific CD8+ T-cells the T-cell is activated, acquiring cytotoxic activity. These cytotoxic T-cells (CTLs) can lyse infected host cells, so limiting the replication and spread of the infecting pathogen. Another important arm of the immune response is controlled by CD4+ T-cells. When antigen derived from pathogens is released into the extracellular milieu they may be taken up by specialised antigen-presenting cells (APCs) and displayed upon the surface of these cells in association with MHC II molecules. Recognition of antigen in this complex stimulates CD4+ T-cells to secrete soluble factors (cytokines) which regulate the effector mechanisms of other T-cells. Antibody is produced by B-cells. Binding of antigen to secreted antibody may neutralise the infectivity of a pathogen and binding of antigen to membrane-bound antibody on the surface of B-cells stimulates division of the B-cell so amplifying the B-cell response. In general, good antibody responses are required to control bacterial infections and both antibody and cell-mediated immune responses (CD8+ and CD4+) are required to control infections by viruses.

It is believed that it may be possible to harness the immune system by vaccination, even after infection by a pathogen, to control or resolve the infection by inactivation or elimination of the pathogen. Such “therapeutic” vaccines would require a cell-mediated response to be effective, and would ideally invoke both humoral and cell-mediated immune responses.

It has been demonstrated (Benvenisty, N and Reshaf, L. PNAS 83 9551-9555) that inoculation of mice with calcium phosphate precipitated DNA results in expression of the peptides encoded by the DNA. Subsequently, intramuscular injection into mice of plasmid DNA which had not been precipitated was shown to result in uptake of the DNA into the muscle cells and expression of the encoded protein. Because expression of the DNA results in production of the encoded pathogen proteins within the host's cells, as in a natural infection, this mechanism can stimulate the cell-mediated immune response required for therapeutic vaccination. DNA vaccines are described in WO90/11092 (Vical, Inc.).

DNA vaccination may be delivered by mechanisms other than intra-muscular injection. For example, delivery into the skin takes advantage of the fact that immune mechanisms are highly active in tissues that are barriers to infection such as skin and mucous membranes. Delivery into skin could be via injection, via jet injector (which forces a liquid into the skin under pressure) or via particle bombardment, in which the DNA may be coated onto particles of sufficient density to penetrate the epithelium (U.S. Pat. No. 5,371,015). Projection of these particles into the skin results in direct transfection of both epidermal cells and epidermal Langerhan cells. Langerhan cells are antigen presenting cells (APC) which take up the DNA, express the encoded peptides, and process these for display on cell surface MHC proteins. Transfected Langerhan cells migrate to the lymph nodes where they present the displayed antigen fragments to lymphocytes, invoking an immune response. Very small amounts of DNA (0.5-1 μg) are required to induce an immune response via particle delivery into skin and this contrasts with the milligram quantities of DNA known to be required to generate immune responses subsequent to direct intramuscular injection.

It has been reported, for example in studies using virus like particles formed from the L1 and L2 capsid proteins or using these proteins alone (1), that HPV is poorly immunogenic. Furthermore, HPV genes have proven difficult to express in human or other mammalian cells, leading difficulties in developing protein subunit vaccines. Monocystronic E1 has proven particularly resistant to expression from heterologous promoters in mammalian cells (J. Virology 1999 73, 3062-3070. Remm M, Remm A and Mart Ustav. Human papilloma virus type 18 E1 is translated from polycistronic mRNA by a discontinuous scanning mechanism). Expression of E1 is most often detected using in vitro DNA replication of an HPV origin containing plasmid as a surrogate (Lu, J Z J, Sun et al J. Virol 1993 67, 7131-7139 and Del Vecchio A M et al J. Virol 1992 66, 5949-5958).

International patent application WO 02/08435 provides HPV polynucleotide wherein the sequence has been optimised to resemble the usage patterns of a highly expressed human gene. In particular codon optimised HPV6bE1, and HPV11 E2 are disclosed.

### BRIEF DESCRIPTION OF THE INVENTION

The present invention provides novel nucleic acid constructs which are useful in the prophylaxis and more particularly in the treatment of the human papillomaviral induced genital warts, or other HPV induced sequalae.

According to a first aspect of the present invention there is provided a nucleic acid construct encoding a polyprotein containing epitopes from at least two distinct Early antigens. Preferably the present invention provides a nucleic acid construct encoding a polyprotein comprising epitopes from three distinct Early antigens. Such construct have been shown by the present inventors to be more efficacious in animal models than the single protein approach.

### DETAILED DESCRIPTION

Preferred constructs include nucleic acids coding for E2 from two different HPV genotypes such as HPV6b and E2 from HPV-11. Additionally it is preferred if an E1 encoding sequence is present. Preferably the E1 is from HPV 6 or 11.

Preferred construct include a nucleic acid molecule having the following arrangement:
1) HPV6bE1-HPV6bE2-HPV11E2
2) HPV6bE2-HPV6bE1-HPV11E2
3) HPV6bE2-HPV11E2-HPV6bE1

Most preferably all the nucleic acid sequence of the above polyprotein has been codon optimised to resemble the codon usage of a highly expressed human gene. Preferably the E1 and E2 genes are substantially full length or more preferably full length. By substantially full length means at least 85% preferably 90% of the E1 and E2 polypeptide is encoded. Surprisingly, such constructs, express to the equivalent expression levels as codon optimised individual proteins, and have the advantage that a single plasmid encoding the polyproteins is cheaper and easier to manufacture than three individual plasmids.

It is preferred that these genes are codon optimised such that the codon usage pattern resembles that of actin, a highly expressed human gene product.

The polynucleotide sequence may be a DNA sequence, for example a double stranded DNA sequence. Preferably the polynucleotide sequence encodes a HPV polypeptide of HPV type 6, 11, 16, 18, 33 or 45, most preferably type 11, sub-type 6a or sub-type 6b. In certain embodiments the encoded amino acid sequence is a wild-type HPV amino acid sequence. In alternative embodiments, the encoded amino acid sequence is a mutated HPV amino acid sequence comprising the wild-type sequence with amino acid changes, for example amino acid point mutations, sufficient to reduce or inactivate one or more of the natural biological functions of the polypeptide. The mutated amino acid sequence will desirably retain the immunogenicity of the wild-type polypeptide.

Proteins encoded by the polynucleotides of the invention also form an aspect of the present invention.

In the case of E1, the primary biological role is to initiate virus specific DNA replication in infected cells. It is preferred that E1 is mutated to inactivate its replication potential.

| | |
|---|---|
| The preferred mutations are: | G 482 D |
| | K 83 G |
| | R 84 G |

Preferably two or more mutations are included.

Most preferably 3 mutations are included.

In the case of E2, this is a site specific binding nuclear protein functioning as the primary replication origin recognition protein and assists in the assembly of the pre-initiation replication complex. It is preferred that the E2 protein is inactivated. A preferred mutation to achieve this objective is K111 A.

According to one aspect of the present invention, the codon usage pattern of the polynucleotide will preferably exclude codons with an RSCU value of less than 0.2 in highly expressed genes in humans. A relative synonymous codon usage (RSCU) value is the observed number of codons divided by the number expected if all codons for that amino acid were used equally frequently. A polynucleotide of the present invention will generally have a codon usage coefficient for highly expressed human genes of greater than 0.3, preferably greater than 0.4, most preferably greater than 0.5. According to a second aspect of the invention, an expression vector is provided which comprises and is capable of directing the expression of a polynucleotide sequence according to the invention, said polynucleotide encoding a polypeptide having epitopes from two or more Early antigens. The vector may be suitable for driving expression of heterologous DNA in bacterial insect or mammalian cells, particularly human cells. In one embodiment, the expression vector is p7313PLc.

In a further aspect, the present invention provides a vaccine composition comprising a protein, or vector, or polynucleotide sequence of the invention. Preferably the vaccine composition comprises a DNA vector according to the present invention. In preferred embodiments the vaccine composition comprises a plurality of particles, preferably gold particles, coated with DNA comprising a vector containing a polynucleotide sequence which encodes a polypeptide having epitopes from two or more Early antigens. In alternative embodiments, the vaccine composition comprises a pharmaceutically acceptable excipient and a DNA vector according to the second aspect of the present invention. The vaccine composition may also include an adjuvant.

In a further aspect, the present invention provides a method of making a vaccine composition including constructing a polynucleotide that encodes a polypeptide that has epitopes from two or more Early antigens and formulating with a pharmaceutically acceptable excipient.

Also provided are the use of a polynucleotide or a vector according to the invention, in the treatment or prophylaxis of an HPV infection, preferably an infection of HPV type 6, 11, 16 or 18. The invention also provides the use of a polynucleotide, a vector according to the invention, in the treatment or prophylaxis of cutaneous (skin) warts, genital warts, atypical squamous cells of undetermined significance (ASCUS), cervical dysplasia, cervical intraepithelial neoplasia (CIN) or cervical cancer. Accordingly, the present invention also provides the use of a polynucleotide or of a vector according to the invention in making a vaccine for the treatment or prophylaxis of an HPV infection or any symptoms or disease associated therewith.

The present invention also provides methods of treating or preventing HPV infections or any symptoms or diseases associated therewith comprising administering an effective amount of a protein, polynucleotide or a vector or a vaccine according to the invention. Administration of a vaccine may take the form of one or more individual doses, for example in a “prime-boost” regime. In certain cases the “prime” vaccination may be via DNA vaccine delivery, in particular via particle mediated DNA delivery of a polynucleotide according to the present invention, preferably incorporated into a plasmid-derived vector and the “boost” by administration of a recombinant viral vector comprising the same polynucleotide sequence. Alternatively, a protein adjuvant approach may act as part of the priming or boosting approach, with DNA delivered as the other arm of the prime-boost regime (the protein being the same as the protein encoded by the DNA).

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

The term “variant” refers to a polynucleotide which encodes the same amino acid sequence as another polynucleotide of the present invention but which, through the redundancy of the genetic code, has a different nucleotide sequence whilst maintaining the same codon usage pattern, for example having the same codon usage coefficient or a codon usage coefficient within 0.1, preferably within 0.05 of that of the other polynucleotide.

The term “codon usage pattern” refers to the average frequencies for all codons in the nucleotide sequence, gene or class of genes under discussion (e.g. highly expressed mammalian genes). Codon usage patterns for mammals, including humans can be found in the literature (see e.g. Nakamura et al. Nucleic Acids Research 1996, 24:214-215).

In the polynucleotides of the present invention, the codon usage pattern is altered from that typical of human papilloma viruses to more closely represent the codon bias of a human. The “codon usage coefficient” is a measure of how closely the codon pattern of a given polynucleotide sequence resembles that of a target species. Codon frequencies can be derived from literature sources for the highly expressed genes of many species (see e.g. Nakamura et al. Nucleic Acids Research 1996, 24:214-215). The codon frequencies for each of the 61 codons (expressed as the number of occurrences occurrence per 1000 codons of the selected class of genes) are normalised for each of the twenty natural amino acids, so that the value for the most frequently used codon for each amino acid is set to 1 and the frequencies for the less common codons are scaled to lie between zero and 1. Thus each of the 61 codons is assigned a value of 1 or lower for the highly expressed genes of the target species. In order to calculate a codon usage coefficient for a specific polynucleotide, relative to the highly expressed genes of that species, the scaled value for each codon of the specific polynucleotide are noted and the geometric mean of all these values is taken (by dividing the sum of the natural logs of these values by the total number of codons and take the anti-log). The coefficient will have a value between zero and 1 and the higher the coefficient the more codons in the polynucleotide are frequently used codons. If a polynucleotide sequence has a codon usage coefficient of 1, all of the codons are “most frequent” codons for highly expressed genes of the target species.

Shorter polynucleotide sequences are within the scope of the invention. For example, a polynucleotide of the invention may encode a fragment of a HPV protein. A polynucleotide which encodes a fragment of at least 8, for example 1-10 amino acids or up to 20, 50, 60, 70, 80, 100, 150 5 or 200 amino acids in length is considered to fall within the scope of the invention as long as the polynucleotide encodes a polypeptide that demonstrates HPV antigenicity. In particular, but not exclusively, this aspect of the invention encompasses the situation when the polynucleotide encodes a fragment of a complete HPV protein sequence and may represent one or more discrete epitopes of that protein.

As discussed above, the present invention includes expression vectors that comprise the nucleotide sequences of the invention. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to persons skilled in the art. By way of further example in this regard we refer to Sambrook et al. Molecular Cloning: a Laboratory Manual. 2^{nd} Edition. CSH Laboratory Press. (1989).

Preferably, a polynucleotide of the invention or for use in the invention in a vector is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be for example, plasmid, artificial chromosome, virus or phage vectors provided with a origin of replication, optionally a promoter for the expression of the said polynucleotide and optionally a regulator of the promoter. The vectors may contain one or more selectable marker genes, for example an ampicillin or kanomycin resistance gene in the case of a bacterial plasmid or a resistance gene for a fungal vector. Vectors may be used in vitro, for example for the production of DNA or RNA or used to transfect or transform a host cell, for example, a mammalian host cell.

The vectors may also be adapted to be used in vivo, for example in a method of DNA vaccination or of gene therapy.

Promoters and other expression regulation signals may be selected to be compatible with the host cell for which expression is designed. For example, mammalian promoters include the metallothionein promoter, which can be induced in response to heavy metals such as cadmium, and the β-actin promoter. Viral promoters such as the SV40 large T antigen promoter, human cytomegalovirus (CMV) immediate early (IE) promoter, rous sarcoma virus LTR promoter, adenovirus promoter), or a HPV promoter, particularly the HPV upstream regulatory region (URR) may also be used. All these promoters are readily available in the art.

Examples of suitable viral vectors include herpes simplex viral vectors, vaccinia or alpha-virus vectors and retroviruses, including lentiviruses, adenoviruses and adeno-associated viruses. Gene transfer techniques using these viruses are known to those skilled in the art. Retrovirus vectors for example may be used to stably integrate the polynucleotide of the invention into the host genome, although such recombination is not preferred. Replication-defective adenovirus vectors by contrast remain episomal and therefore allow transient expression. Vectors capable of driving expression in insect cells (for example baculovirus vectors), in human cells or in bacteria may be employed in order to produce quantities of the HPV protein encoded by the polynucleotides of the present invention, for example for use as subunit vaccines. Preferred viral vectors are those derived from non-human primate adenovirus such as C68 chimp adenovirus (U.S. Pat. No. 6,083,716) other wise known as Pan 9.

Where the polynucleotides of the present invention find use as therapeutic agents, e.g. in DNA vaccination, the nucleic acid will be administered to the mammal e.g. human to be vaccinated. The nucleic acid, such as RNA or DNA, preferably DNA, is provided in the form of a vector, such as those described above, which may be expressed in the cells of the mammal. The polynucleotides may be administered by any available technique. For example, the nucleic acid may be introduced by needle injection, preferably intradermally, subcutaneously or intramuscularly. Alternatively, the nucleic acid may be delivered directly across the skin using a nucleic acid delivery device such as particle-mediated DNA delivery (PMDD). In this method, inert particles (such as gold beads) are coated with a nucleic acid, and are accelerated at speeds sufficient to enable them to penetrate a surface of a recipient (e.g. skin), for example by means of discharge under high pressure from a projecting device. (Particles coated with a nucleic acid molecule of the present invention are within the scope of the present invention, as are devices loaded with such particles).

Suitable techniques for introducing the naked polynucleotide or vector into a patient include topical application with an appropriate vehicle. The nucleic acid may be administered topically to the skin, or to mucosal surfaces for example by intranasal, oral, intravaginal or intrarectal administration. The naked polynucleotide or vector may be present together with a pharmaceutically acceptable excipient, such as phosphate buffered saline (PBS). DNA uptake may be further facilitated by addition of facilitating agents such as bupivacaine to the composition. Other methods of administering the nucleic acid directly to a recipient include ultrasound, electrical stimulation, electroporation and microseeding which is described in U.S. Pat. No. 5,697,901.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered. Typically the nucleic acid is administered in an amount in the range of 1 pg to 1 mg, preferably to 1 pg to 10 μg nucleic acid for particle mediated gene delivery and 10 μg to 1 mg for other routes.

A nucleic acid sequence of the present invention may also be administered by means of specialised delivery vectors useful in gene therapy. Gene therapy approaches are discussed for example by Verme et al, Nature 1997, 389:239-242. Both viral and non-viral systems can be used. Viral based systems include retroviral, lentiviral, adenoviral, adeno-associated viral, herpes viral, Canarypox and vaccinia-viral based systems. Non-viral based systems include direct administration of nucleic acids and liposome-based systems.

A nucleic acid sequence of the present invention may also be administered by means of transformed cells. Such cells include cells harvested from a subject. The naked polynucleotide or vector of the present invention can be introduced into such cells in vitro and the transformed cells can later be returned to the subject. The polynucleotide of the invention may integrate into nucleic acid already present in a cell by homologous recombination events. A transformed cell may, if desired, be grown up in vitro and one or more of the resultant cells may be used in the present invention. Cells can be provided at an appropriate site in a patient by known surgical or microsurgical techniques (e.g. grafting, micro-injection, etc.)

The vaccine compositions of the present invention may include adjuvant compounds which may serve to increase the immune response induced by the protein itself or which is encoded by the plasmid DNA. Alteration of the codon bias to suit the vaccinated species is proposed herein as a means of increasing expression and thereby boosting the immune response, but an adjuvant may never-the-less be desirable because, while DNA vaccines tend to work well in mice models, there is evidence of a somewhat weaker potency in larger species such as non-human primates which is thought to be predictive of the likely potency in humans.

The vaccine composition of the invention may also comprise an adjuvant, such as, for example, in an embodiment, imiquimod, tucaresol or alum.

Preferably the adjuvant is administered at the same time as of the invention and in preferred embodiments are formulated together. Such adjuvant agents contemplated by the invention include, but this list is by no means exhaustive and does not preclude other agents: synthetic imidazoquinolines such as imiquimod [S-26308, R-837], (Harrison, et al. ‘Reduction of recurrent HSV disease using imiquimod alone or combined with a glycoprotein vaccine’, Vaccine 19: 1820-1826, (2001)); and resiquimod [S-28463, R-848] (Vasilakos, et al. ‘Adjuvant activates of immune response modifier R-848: Comparison with CpG ODN’, Cellular immunology 204: 64-74 (2000).), Schiff bases of carbonyls and amines that are constitutively expressed on antigen presenting cell and T-cell surfaces, such as tucaresol (Rhodes, J. et al. ‘Therapeutic potentiation of the immune system by costimulatory Schiff-base-forming drugs’, Nature 377: 71-75 (1995)), cytokine, chemokine and co-stimulatory molecules, Th1 inducers such as interferon gamma, IL-2, IL-12, IL-15 and IL-18, Th2 inducers such as IL-4, IL-5, IL-6, IL-10 and IL-13 and other chemokine and co-stimulatory genes such as MCP-1, MIP-1 alpha, MIP-1 beta, RANTES, TCA-3, CD80, CD86 and CD40L, other immunostimulatory targeting ligands such as CTLA-4 and L-selectin, apoptosis stimulating proteins and peptides such as Fas, (49), synthetic lipid based adjuvants, such as vaxfectin, (Reyes et al., ‘Vaxfectin enhances antigen specific antibody titres and maintains Th1 type immune responses to plasmid DNA immunization’, Vaccine 19: 3778-3786) squalene, alpha-tocopherol, polysorbate 80, DOPC and cholesterol, endotoxin, [LPS], Beutler, B., ‘Endotoxin, ‘Toll-like receptor 4, and the afferent limb of innate immunity’, Current Opinion in Microbiology 3: 23-30 (2000)); CpG oligo- and di-nucleotides, Sato, Y. et al., ‘Immunostimulatory DNA sequences necessary for effective intradermal gene immunization’, Science 273 (5273): 352-354 (1996). Hemmi, H. et al., ‘A Toll-like receptor recognizes bacterial DNA’, Nature 408: 740-745, (2000) and other potential ligands that trigger Toll receptors to produce Th1-inducing cytokines, such as synthetic Mycobacterial lipoproteins, Mycobacterial protein p19, peptidoglycan, teichoic acid and lipid A.

Certain preferred adjuvants for eliciting a predominantly Th1-type response include, for example, a Lipid A derivative such as monophosphoryl lipid A, or preferably 3-de-O-acylated monophosphoryl lipid A. MPL® adjuvants are available from Corixa Corporation (Seattle, Wash.; see, for example, U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins.

In an embodiment, the adjuvant comprises an immunostimulatory CpG oligonucleotide, such as disclosed in (WO96102555). Typical immunostimulatory oligonucleotides will be between 8-100 bases in length and comprises the general formula X₁ CpGX₂ where X₁ and X₂ are nucleotide bases, and the C and G are unmethylated.

The preferred oligonucleotides for use in adjuvants or vaccines of the present invention preferably contain two or more dinucleotide CpG motifs preferably separated by at least three, more preferably at least six or more nucleotides. The oligonucleotides of the present invention are typically deoxynucleotides. In a preferred embodiment the internucleotide in the oligonucleotide is phosphorodithioate, or more preferably a phosphorothioate bond, although phosphodiester and other internucleotide bonds are within the scope of the invention including oligonucleotides with mixed internucleotide linkages. e.g. mixed phosphorothioate/phophodiesters. Other internucleotide bonds which stabilise the oligonucleotide may be used. Methods for producing phosphorothioate oligonucleotides or phosphorodithioate are described in U.S. Pat. No. 5,666,153, U.S. Pat. No. 5,278,302 and WO95/26204.

Examples of preferred oligonucleotides have the following sequences. The sequences preferably contain phosphorothioate modified internucleotide linkages.

OLIGO 1:

(SEQ ID NO 24)

TCC ATG ACG TTC CTG ACG TT (CpG 1826)
OLIGO 2:

(SEQ ID NO 25)

TCT CCC AGC GTG CGC CAT (CpG 1758)
OLIGO 3:

(SEQ ID NO 26)

ACC GAT GAC GTC GCC GGT GAC GGC ACC ACG
OLIGO 4:

(SEQ ID NO 27)

TCG TCG TTT TGT CGT TTT GTC GTT (CpG 2006)
OLIGO 5:

(SEQ ID NO 28)

TCC ATG ACG TTC CTG ATG CT (CpG 1668)

Alternative CpG oligonucleotides may comprise the preferred sequences above in that they have inconsequential deletions or additions thereto.

The CpG oligonucleotides utilised in the present invention may be synthesized by any method known in the art (eg EP 468520). Conveniently, such oligonucleotides may be synthesized utilising an automated synthesizer. An adjuvant formulation containing CpG oligonucleotide can be purchased from Qiagen under the trade name “ImmunEasy”.

The following Examples serve to further illustrate the invention, with reference to the accompanying drawings, in which:

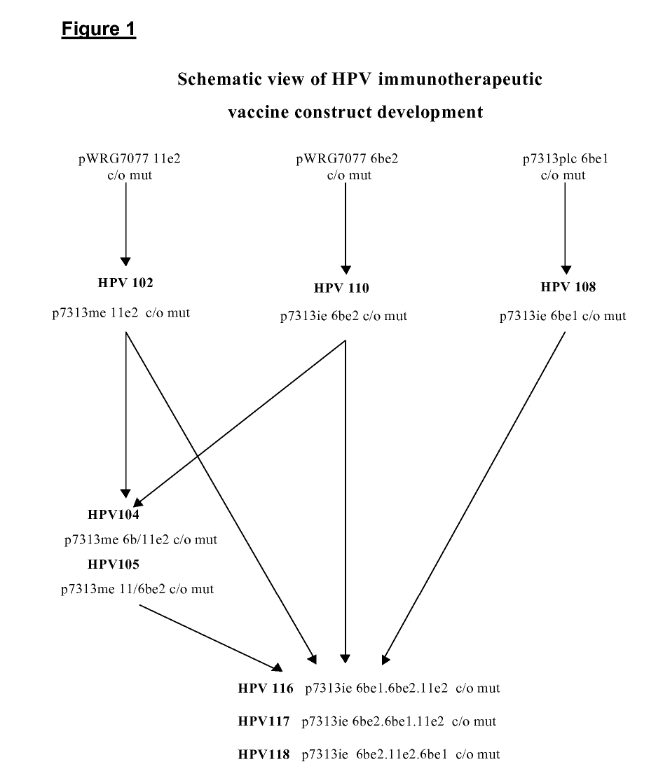

FIG. 1 is a schematic view of HPV Immunotherapeutic vaccine construct of the invention.

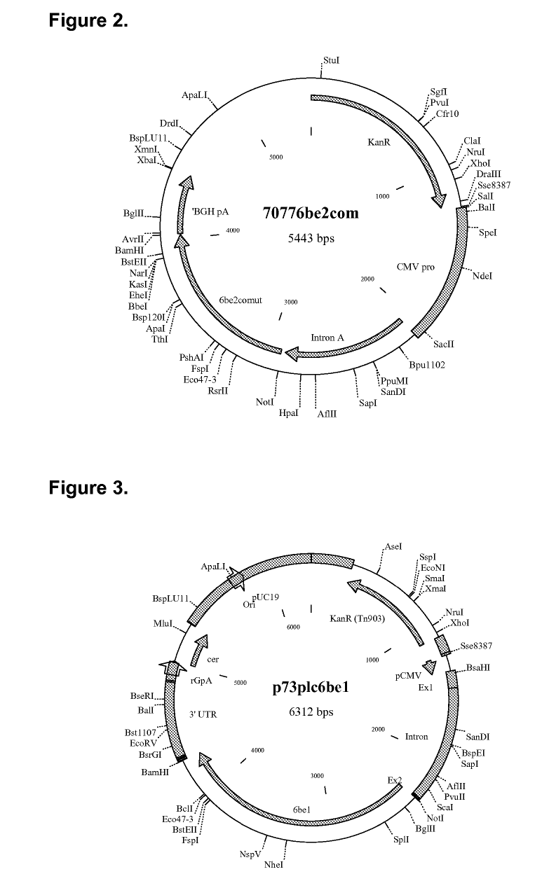

FIG. 2 is a plasmid map of P70776be2—encoding HPV 6b E2 that has been codon optimised and 20 mutated.

FIG. 3 is a plasmid map of p73p1c6be1—encoding HPV 6b E1 that has been codon optimised and mutated.

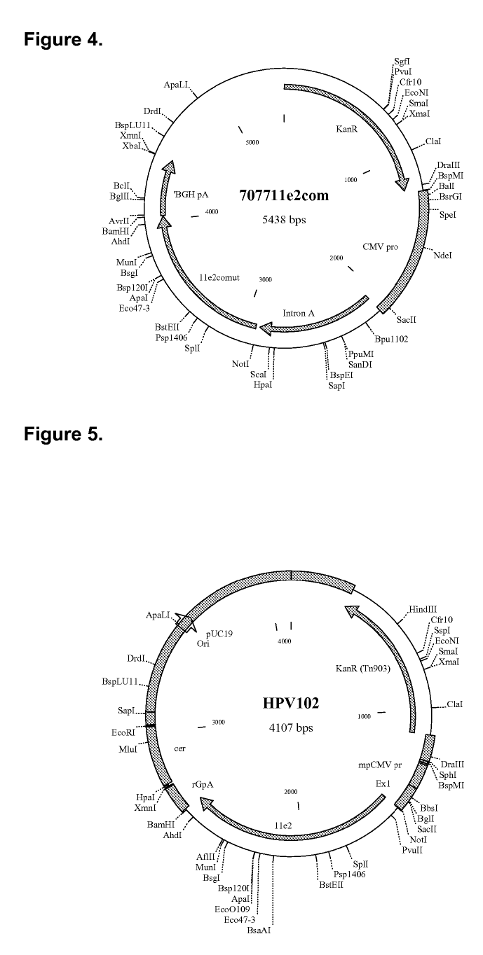

FIG. 4 is a plasmid map of p707711e2—encoding HPV 11 E2 that has been codon optimised and mutated.

FIG. 5 is a plasmid map of HPV 102—encoding HPV 11 E2 in p7313 background.

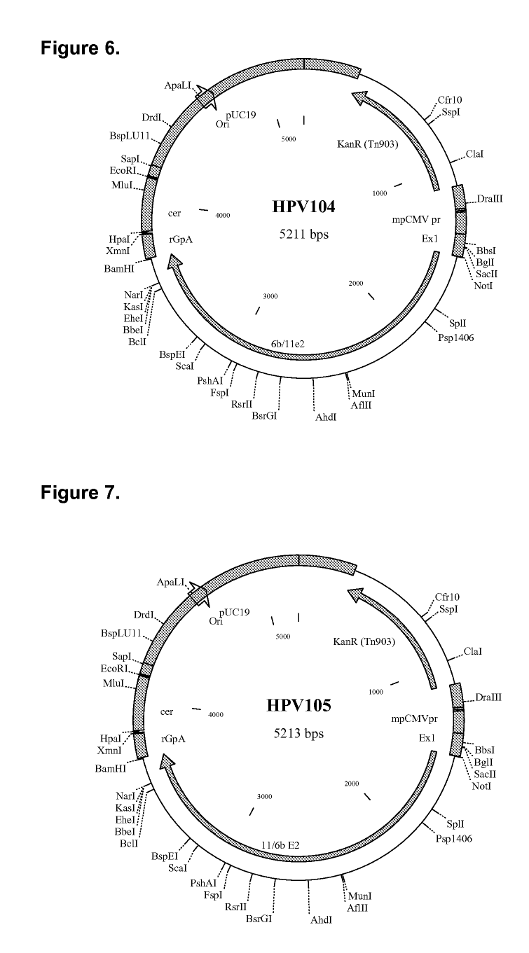

FIG. 6 is a plasmid map of HPV 104—fusion of E2 from HPV 6b and E2 from HPV 11 in p7313 background.

FIG. 7 is a plasmid map of HPV 105—fusion of codon optimised, mutated HPV 6b E2 and E2 from HPV 11.

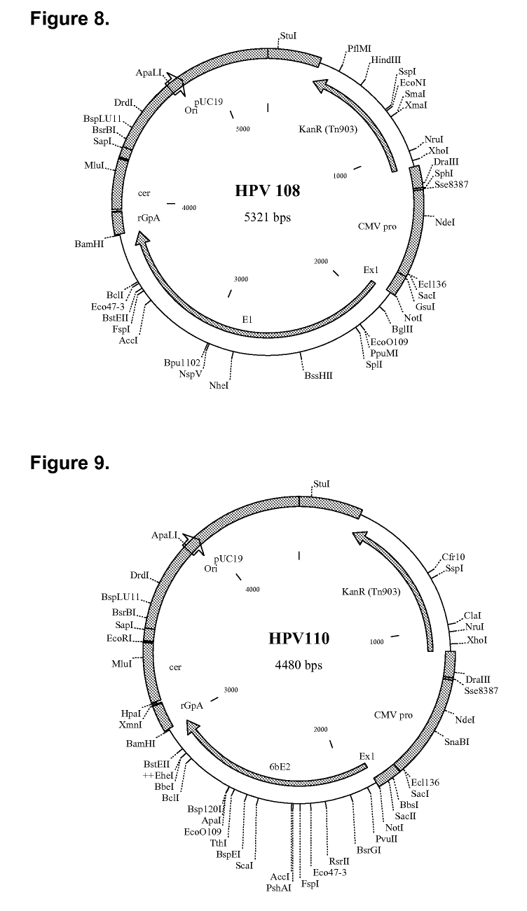

FIG. 8 is a plasmid map of HPV 108—HPV 6b E1 codon optimised, mutated in p7313 background.

FIG. 9 is a plasmid map of HPV 110—HPV 6b E2 codon optimised, mutated in p7313 background.

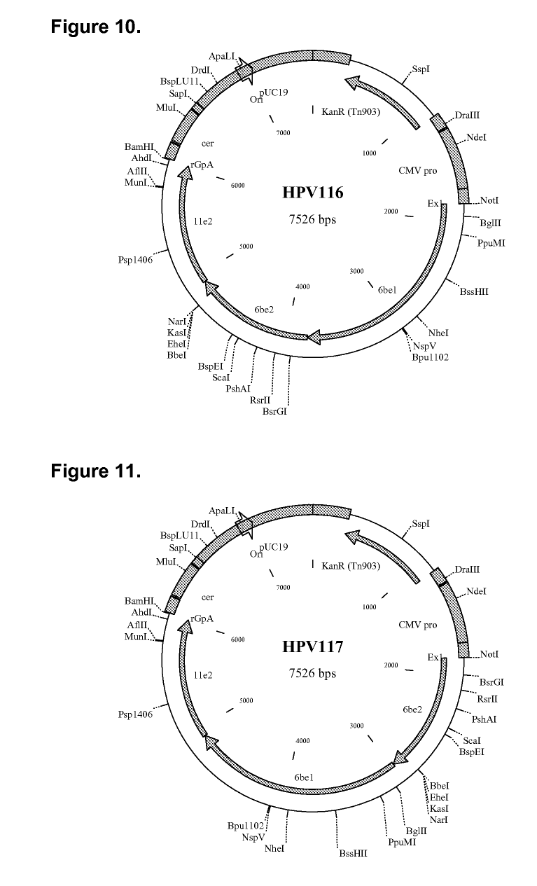

FIG. 10 is a plasmid map of HPV 116—HPV 6b E1, HPV 6b E2, HPV 11 E2.

FIG. 11 is a plasmid map of HPV 117—HPV 6b E2, HPV 11 E2, HPV 6b E1.

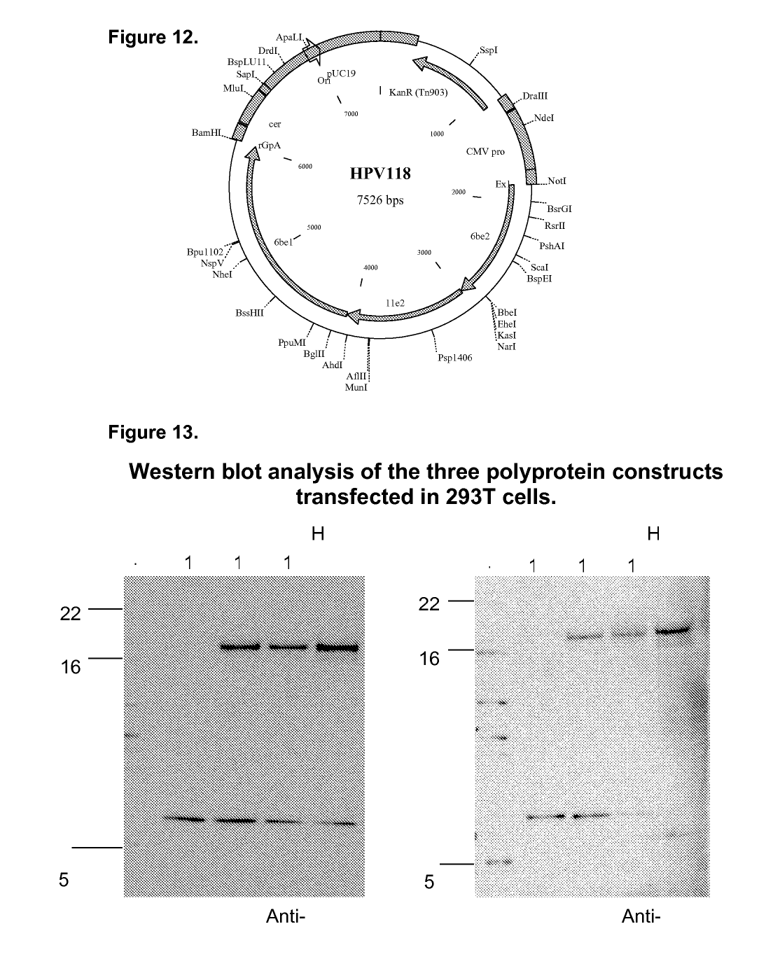

FIG. 12 is a plasmid map of HPV 118—HPV 6b E2, HPV 11 E2, HPV 6b E1.

FIG. 13 is a western blot analysis of three polyprotein constructs of the invention in 293 T cells.

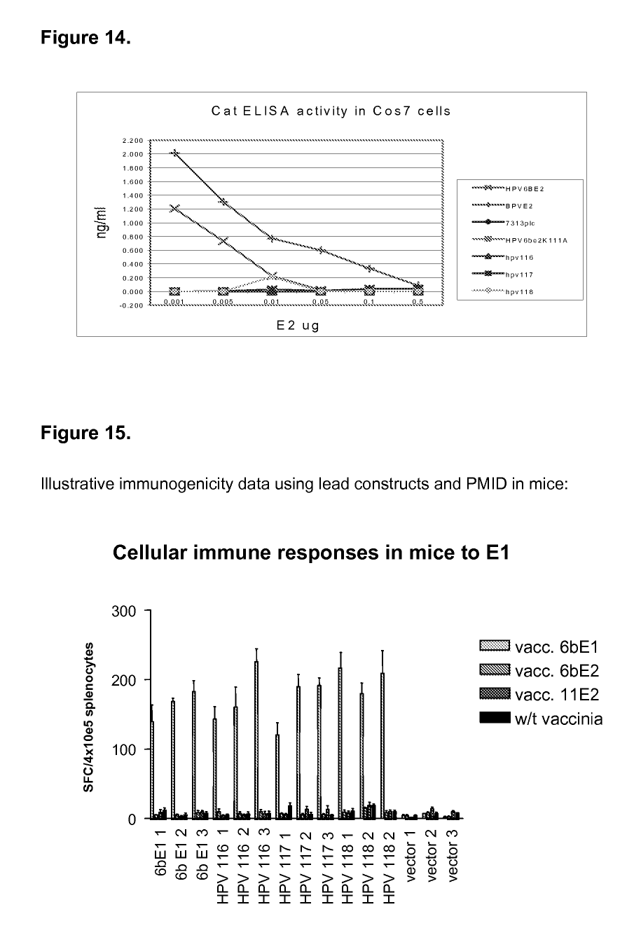

FIG. 14 shows the incapacity of K111A mutated E2 in an in vitro CAT transcriptional reporter assay.

FIG. 15 shows cellular immune response in mice to E1

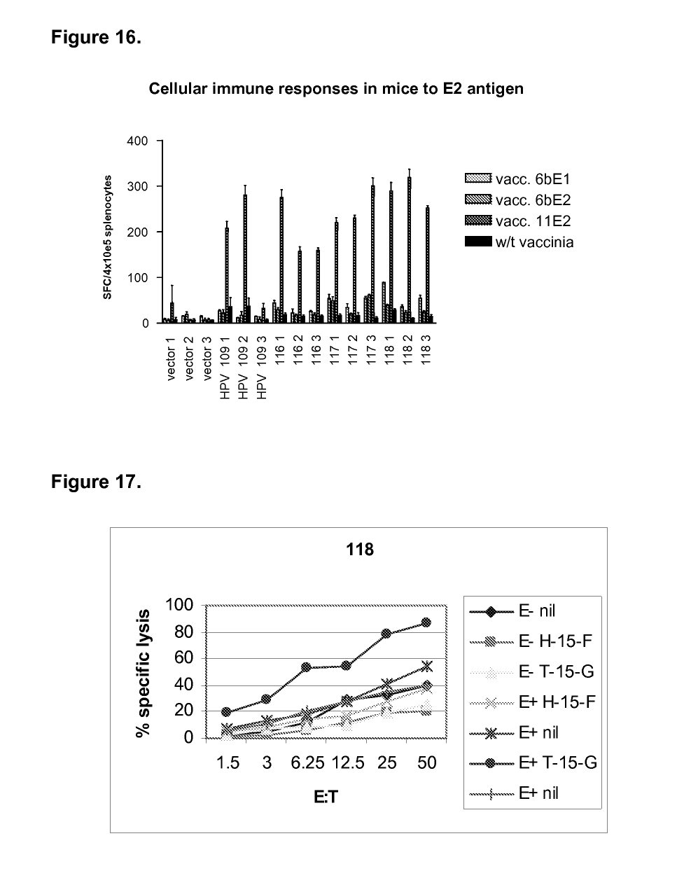

FIG. 16 shows cellular immune response to E2

FIG. 17—CTL assay data with HPV 118 after PMID

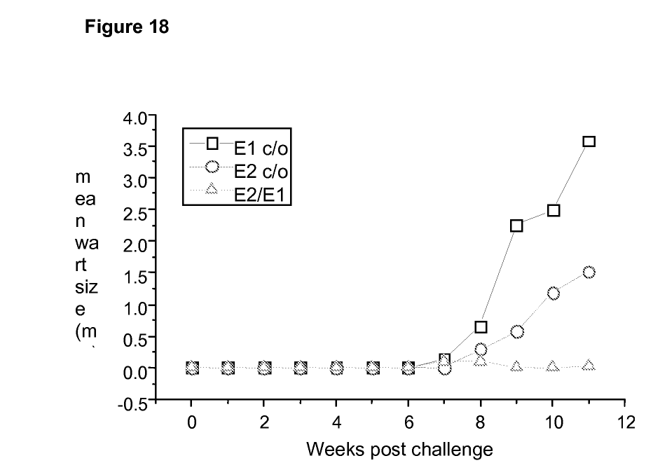

FIG. 18 shows reduction of warts after administration of E1/E2 in the COPV model.

### 1. PLASMID: pWRG7077 6be2 c/o MUTATED

Gene of Interest:

The HPV6be2 gene is approximately 1.1 Kb in size and a codon optimised sequence (for human expression) was created using a visual basic programme called Syngene. In addition the sequence included a codon change at amino acid position 111, whereby a lysine residue (AAG) in the wild type was changed to an alanine residue (GCA) creating a mutated gene. This change inactivates the transcriptional activity of 6be2. Overlapping primers incorporating the whole gene with selected restriction sites at both the 5′ and 3′ ends were designed accordingly.

Cloning:

The 1.1 kb PCR fragment was gel purified and digested with restriction enzymes Not I and Bam HI for ligation into vector pWRG7077 (Powderject). The gene is under control of the full immediate early CMV promoter and have a bovine growth hormone poly A tail.

Clones were sequenced indicated a number of base errors. A number of suitable clones were identified to enable construction of the correct gene sequence by using restriction digests. From re-cloning, one clone C7 was found to have only one base error at position 497 (T to C). Other clones were o.k. in this area and a simple fragment swap was just needed to correct the error. The final clone C7a was confirmed to be codon optimised mutated 6be2. (See FIG. 2)

6be2 sequence in pWRG7077

(Sequence ID No. 1)

ATGGAAGCTATTGCCAAGCGACTGGACGCCTGCCAGGAGCAGCTGCTGGA
GCTGTACGAGGAAAACAGCACAGACCTCCACAAGCACGTGCTGCACTGGA
AGTGCATGCGCCACGAGTCAGTGCTCCTGTACAAGGCCAAGCAGATGGGG
CTGTCCCACATCGGGATGCAGGTCGTGCCCCCGCTGAAGGTGAGCGAAGC
CAAGGGCCACAACGCTATCGAGATGCAGATGCACCTGGAGAGCCTGCTGC
GGACCGAATACAGCATGGAGCCCTGGACTCTCCAGGAGACGTCCTACGAA
ATGTGGCAGACTCCTCCGAAGCGCTGTTTCGCAAAGCGCGGCAAGACAGT
TGAGGTGAAATTCGATGGGTGCGCAAACAACACGATGGACTACGTGGTGT
GGACCGATGTCTACGTGCAGGACAATGACACCTGGGTGAAGGTACATAGT
ATGGTGGATGCCAAGGGCATCTATTACACCTGCGGGCAGTTCAAGACGTA
CTACGTCAACTTCGTCAAGGAAGCCGAAAAGTATGGTTCCACCAAGCACT
GGGAGGTGTGTTACGGGAGTACTGTGATCTGCAGCCCCGCCTCCGTGTCG
TCCACCACCCAGGAAGTGAGCATTCCGGAGAGCACCACATACACCCCGGC
CCAAACGAGCACGCTCGTCAGCAGCAGCACCAAGGAGGACGCCGTCCAGA
CGCCCCCCCGGAAGAGGGCCCGGGGGGTCCAGCAGTCTCCCTGCAATGCC
CTGTGCGTTGCTCACATCGGCCCTGTCGATTCTGGGAACCACAATCTCAT
CACGAACAACCACGACCAGCACCAAAGGCGCAACAACTCTAACAGCTCCG
CAACTCCAATAGTGCAGTTCCAGGGGGAGTCCAACTGCCTCAAGTGTTTC
CGCTACCGCCTCAACGACCGCCACCGCCACCTGTTCGACTTGATCAGTTC
CACGTGGCACTGGGCCAGCAGCAAGGCGCCCCACAAACACGCTATCGTGA
CGGTGACCTACGACTCCGAGGAGCAGAGGCAGCAGTTCCTGGACGTCGTG
AAGATTCCTCCGACAATCAGCCACAAGCTTGGCTTCATGTCCCTGCACCT
GCTGTGA
Amino acid sequence

(Seq. ID No. 2)

MEAIAKRLDA CQEQLLELYE ENSTDLHKHV LHWKCMRHES
VLLYKAKQMG LSHIGMQVVP PLKVSEAKGH NAIEMQMHLE
SLLRTEYSME PWTLQETSYE MWQTPPKRCF AKRGKTVEVK
FDGCANNTMD YVVWTDVYVQ DNDTWVKVHS MVDAKGIYYT
CGQFKTYYVN FVKEAEKYGS TKHWEVCYGS TVICSPASVS
STTQEVSIPE STTYTPAQTS TLVSSSTKED AVQTPPRKRA
RGVQQSPCNA LCVAHIGPVD SGNHNLITNN HDQHQRRNNS
NSSATPIVQF QGESNCLKCF RYRLNDRHRH LFDLISSTWH
WASSKAPHKH AIVTVTYDSE EQRQQFLDVV KIPPTISHKL
GFMSLHLL

### 2. PLASMID: p7313plc 6be1 c/o mut

Gene of Interest:

The HPV6be1 gene is approximately 2 Kb in size and a codon optimised wild type (wt) sequence (for E. coli and human expression) was created using a statistical visual basic programme called Syngene. Overlapping primers incorporating the whole gene with selected restriction sites at both the 5′ and 3′ ends were designed accordingly. The synthesised gene was then digested with Bam HI and Not I restriction enzymes for ligation into vector pCIN4. From the sequencing data for a number of selected clones, numerous base errors were discovered. A correct clone was generated by combining a correct Pst I-Bam HI fragment from clone #24 and a Not I-Pst I fragment from clone #21 into p7313-plc. A correct clone (#1) was confirmed by sequencing. For mutagenesis primers were designed to change the following amino acids; lysine (AAA) to glycine (GGA) at position 83, arginine (CGC) to glycine (GGC) at position 84 and glycine (GGC) to asparagine (GAC) at position 482.

6be1 codon optimised mutated sequence

(Seq ID No. 3)

ATGGCAGACGATTCCGGTACTGAGAACGAAGGTTCTGGTTGTACCGGTTG
GTTCATGGTTGAAGCAATCGTTCAGCATCCGACTGGTACCCAGATCTCCG
ATGACGAAGACGAAGAAGTTGAAGATTCTGGTTACGACATGGTTGACTTC
ATCGATGACTCCAACATCACTCATAACTCTCTGGAAGCACAGGCTCTGTT
TAACCGCCAGGAAGCTGATACCCATTACGCTACTGTTCAGGACCTGGGAG
GCAAATATCTGGGCTCTCCGTACGTTTCCCCGATCAACACTATCGCAGAA
GCAGTTGAGTCTGAAATCTCCCCGCGCCTGGACGCTATCAAACTGACTCG
TCAGCCGAAGAAGGTTAAACGTCGTCTGTTCCAGACTCGTGAACTGACCG
ACTCCGGTTACGGTTATAGCGAAGTTGAGGCTGGCACCGGCACCCAGGTT
GAAAAACACGGTGTACCGGAAAACGGCGGCGACGGTCAGGAAAAGGACAC
CGGCCGCGACATCGAGGGTGAGGAACACACCGAAGCTGAAGCTCCGACTA
ACTCTGTTCGTGAACACGCAGGTACTGCGGGTATCCTGGAACTGCTGAAA
TGCAAAGACCTGCGCGCGGCTCTGCTGGGCAAATTCAAAGAATGCTTCGG
CCTGTCTTTCATTGACCTGATCCGTCCGTTTAAGTCTGACAAAACTACCT
GTCTGGACTGGGTTGTAGCAGGCTTCGGCATCCACCACTCTATCTCTGAA
GCATTCCAGAAACTGATCGAGCCGCTGTCTCTGTACGCGCACATCCAGTG
GCTGACTAACGCTTGGGGTATGGTTCTGCTGGTACTGCTGCGCTTTAAAG
TAAACAAATCTCGTTCCACTGTTGCTCGTACTCTGGCTACCCTGCTGAAC
ATCCCGGAGAACCAGATGCTGATCGAACCGCCGAAAATCCAGTCTGGTGT
AGCTGCACTGTACTGGTTTCGTACTGGCATCTCTAACGCTAGCACTGTTA
TCGGTGAAGCACCGGAATGGATCACTCGTCAGACCGTTATCGAACACGGT
CTGGCAGATTCTCAGTTCAAACTGACTGAAATGGTTCAGTGGGCATACGA
CAACGACATCTGCGAGGAATCTGAAATTGCGTTCGAATACGCTCAGCGTG
GCGACTTCGACTCCAACGCTCGTGCTTTCCTGAACAGCAACATGCAGGCT
AAATACGTAAAAGACTGCGCTACCATGTGCCGTCACTACAAACACGCGGA
AATGCGTAAAATGTCTATCAAACAGTGGATCAAGCACCGCGGTTCTAAAA
TCGAAGGTACCGGTAACTGGAAACCGATCGTTCAGTTCCTGCGCCATCAG
AACATCGAATTCATCCCGTTCCTGACCAAATTCAAGCTGTGGCTGCACGG
TACCCCGAAAAAAAACTGCATCGCTATCGTAGGTCCACCGGACACTGACA
AGTCTTACTTCTGTATGTCCCTGATCTCTTTCCTGGGCGGCACTGTAATC
TCTCACGTTAACTCTTCCTCCCATTTCTGGCTGCAGCCACTGGTAGACGC
GAAAGTAGCTCTGCTGGACGACGCGACCCAGCCGTGCTGGATCTACATGG
ATACTTACATGCGCAACCTGCTGGACGGTAACCCGATGTCTATCGACCGT
AAACACAAAGCGCTGACTCTGATCAAGTGCCCGCCGCTGCTGGTAACTTC
TAACATCGACATCACCAAGGAAGATAAATACAAGTACCTGCATACCCGTG
TTACTACCTTTACTTTCCCGAACCCGTTCCCGTTTGATCGTAACGGTAAC
GCTGTTTACGAACTGTCCAACACTAACTGGAAATGCTTCTTCGAGCGTCT
GTCTTCCTCCCTGGACATCCAGGACTCTGAAGATGAAGAAGATGGTTCTA
ACTCTCAGGCTTTCCGTTGTGTTCCGGGTACTGTTGTTCGTACTCTGTGA
Amino acid sequence

(Seq. ID No. 4)

MADDSGTENE GSGCTGWFMV EAIVQHPTGT QISDDEDEEV
EDSGYDMVDF IDDSNITHNS LEAQALFNRQ EADTHYATVQ
DLGGKYLGSP YVSPINTIAE AVESEISPRL DAIKLTRQPK
KVKRRLFQTR ELTDSGYGYS EVEAGTGTQV EKHGVPENGG
DGQEKDTGRD IEGEEHTEAE APTNSVREHA GTAGILELLK
CKDLRAALLG KFKECFGLSF IDLIRPFKSD KTTCLDWVVA
GFGIHHSISE AFQKLIEPLS LYAHIQWLTN AWGMVLLVLL
RFKVNKSRST VARTLATLLN IPENQMLIEP PKIQSGVAAL
YWFRTGISNA STVIGEAPEW ITRQTVIEHG LADSQFKLTE
MVQWAYDNDI CEESEIAFEY AQRGDFDSNA RAFLNSNMQA
KYVKDCATMC RHYKHAEMRK MSIKQWIKHR GSKIEGTGNW
KPIVQFLRHQ NIEFIPFLTK FKLWLHGTPK KNCIAIVGPP
DTDKSYFCMS LISFLGGTVI SHVNSSSHFW LQPLVDAKVA
LLDDATQPCW IYMDTYMRNL LDGNPMSIDR KHKALTLIKC
PPLLVTSNID ITKEDKYKYL HTRVTTFTFP NPFPFDRNGN
AVYELSNTNW KCFFERLSSS LDIQDSEDEE DGSNSQAFRC
VPGTVVRTL

### 3. PLASMID: WRG7077 11e2 c/o mut

Gene of Interest:

The HPV11e2 gene is approximately 1.1 Kb in size and a codon optimised sequence (for human expression) was created using a visual basic programme called Syngene. In addition the sequence included a codon change at amino acid position 111, whereby a lysine residue (AAG) in the wild type was changed to an alanine residue (GCC) creating a mutated gene. This change has been shown in the literature to inactivate the transcriptional activity of the E2 protein. Overlapping primers incorporating the whole gene with selected restriction sites at both the 5′ and 3′ ends were designed accordingly, and were used to assemble the synthetic codon optimised mutant 11e2.

Cloning:

The 1.2 kb PCR fragment was gel purified and digested with restriction enzymes Not I and Bam HI for ligation into vector pWRG7077 (Powderject). The gene is under control of the full immediately early CMV promoter and has a bovine growth hormone poly A tail.

Clones that were sequenced had indicated a number of base errors, these were subsequently corrected. A final clone F1 was found to be codon optimised mutated 11E2.

11e2 sequence in pWRG7077

(Seq. ID No. 5)

ATGGAAGCCATCGCGAAGAGGCTCGACGCCTGCCAGGACCAGCTGCTCGA
GCTGTACGAGGAGAACAGCATTGACATCCATAAGCACATCATGCACTGGA
AGTGCATTCGCCTGGAGAGCGTGCTGTTGCACAAGGCCAAGCAGATGGGC
CTGTCCCACATAGGCCTTCAGGTGGTCCCCCCTCTGACCGTGTCAGAGAC
AAAGGGCCATAACGCAATCGAGATGCAGATGCACCTCGAGTCGCTGGCGA
AAACACAGTACGGCGTGGAGCCATGGACCCTCGAGGACACCTCGTACGAA
ATGTGGCTGACCCCACCTAAGCGATGCTTCGCCAAACAGGGCAACACAGT
GGAGGTGAAGTTCGACGGCTGTGAGGATAACGTTATGGAGTATGTCGTGT
GGACGCACATCTATCTGCAGGACAACGACAGTTGGGTGAAGGTGACCAGC
TCCGTGGACGCGAAGGGCATCTACTATACCTGTGGGCAGTTTAAAACCTA
CTATGTGAACTTCAACAAAGAGGCCCAAAAGTATGGCTCCACCAACCACT
GGGAGGTCTGCTATGGGAGCACGGTGATTTGCTCTCCCGCCAGCGTGTCT
AGCACTGTGCGCGAGGTGAGCATTGCCGAGCCGACCACGTACACCCCTGC
CCAGACGACCGCTCCGACCGTGTCTGCTTGTACTACCGAGGACGGCGTGA
GCGCTCCACCCAGGAAGCGTGCGAGGGGCCCAAGCACCAACAACACCCTC
TGTGTGGCGAACATTCGCAGCGTCGACAGTACCATCAATAACATCGTGAC
GGATAACTATAACAAGCACCAGAGGCGTAACAACTGTCACTCTGCCGCAA
CCCCCATCGTGCAGCTCCAGGGAGACAGCAATTGCCTTAAGTGCTTCCGC
TATCGCCTCAACGACAAGTACAAGCACCTCTTTGAGCTCGCCTCGTCGAC
GTGGCACTGGGCCTCACCCGAGGCACCTCACAAGAACGCCATCGTCACTC
TCACTTACTCCAGTGAGGAGCAGAGACAGCAGTTTCTGAACAGCGTGAAG
ATCCCACCGACGATCCGTCATAAGGTCGGCTTCATGTCACTGCATCTCCT
GTGA
Amino acid sequence

(Seq. ID No. 6)

MEAIAKRLDA CQDQLLELYE ENSIDIHKHI MHWKCIRLES
VLLHKAKQMG LSHIGLQVVP PLTVSETKGH NAIEMQMHLE
SLAKTQYGVE PWTLQDTSYE MWLTPPKRCF AKQGNTVEVK
FDGCEDNVME YVVWTHIYLQ DNDSWVKVTS SVDAKGIYYT
CGQFKTYYVN FNKEAQKYGS TNHWEVCYGS TVICSPASVS
STVREVSIAE PTTYTPAQTT APTVSACTTE DGVSAPPRKR
ARGPSTNNTL CVANIRSVDS TINNIVTDNY NKHQRRNNCH
SAATPIVQLQ GDSNCLKCFR YRLNDKYKHL FELASSTWHW
ASPEAPHKNA IVTLTYSSEE QRQQFLNSVK IPPTIRHKVG
FMSLHLL

### 4. PLASMID: HPV102 (p7313me 11e2 c/o mut)

Gene of Interest:

Codon optimised mutated 11e2 was transferred from pWRG7077 11e2 c/o mut into another expression vector p7313me.

Cloning:

The 11e2 c/o mut fragment was cut out of pWRG7077 11e2 vector by Bam HI and Not I restriction enzymes. This fragment was then ligated into p7313me vector using these sites.

11e2 sequence in HPV102

(Seq. ID No. 7)

ATGGAAGCCATCGCGAAGAGGCTCGACGCCTGCCAGGACCAGCTGCTCGA
GCTGTACGAGGAGAACAGCATTGACATCCATAAGCACATCATGCACTGGA
AGTGCATTCGCCTGGAGAGCGTGCTGTTGCACAAGGCCAAGCAGATGGGC
CTGTCCCACATAGGCCTTCAGGTGGTCCCCCCTCTGACCGTGTCAGAGAC
AAAGGGCCATAACGCAATCGAGATGCAGATGCACCTCGAGTCGCTGGCGA
AAACACAGTACGGCGTGGAGCCATGGACCCTGCAGGACACCTCGTACGAA
ATGTGGCTGACCCCACCTAAGCGATGCTTCGCCAAACAGGGCAACACAGT
GGAGGTGAAGTTCGACGGCTGTGAGGATAACGTTATGGAGTATGTCGTGT
GGACGCACATCTATCTGCAGGACAACGACAGTTGGGTGAAGGTGACCAGC
TCCGTGGACGCGAAGGGCATCTACTATACCTGTGGGCAGTTTAAAACCTA
CTATGTGAACTTCAACAAAGAGGCCCAAAAGTATGGCTCCACCAACCACT
GGGAGGTCTGCTATGGGAGCACGGTGATTTGCTCTCCCGCCAGCGTGTCT
AGCACTGTGCGCGAGGTGAGCATTGCCGAGCCGACCACGTACACCCCTGC
CCAGACGACCGCTCCGACCGTGTCTGCTTGTACTACCGAGGACGGCGTGA
GCGCTCCACCCAGGAAGCGTGCGAGGGGCCCAAGCACCAACAACACCCTC
TGTGTGGCGAACATTCGCAGCGTCGACAGTACCATCAATAACATCGTGAC
GGATAACTATAACAAGCACCAGAGGCGTAACAACTGTCACTCTGCCGCAA
CCCCCATCGTGCAGCTCCAGGGAGACAGCAATTGCCTTAAGTGCTTCCGC
TATCGCCTCAACGACAAGTACAAGCACCTCTTTGAGCTCGCCTCGTCGAC
GTGGCACTGGGCCTCACCCGAGGCACCTCACAAGAACGCCATCGTCACTC
TCACTTACTCCAGTGAGGAGCAGAGACAGCAGTTTCTGAACAGCGTGAAG
ATCCCACCGACGATCCGTCATAAGGTCGGCTTCATGTCACTGCATCTCCT
GTGA
Amino acid sequence

(Seq. ID No. 8)

MEAIAKRLDA CQDQLLELYE ENSIDIHKHI MHWKCIRLES
VLLHKAKQMG LSHIGLQVVP PLTVSETKGH NAIEMQMHLE
SLAKTQYGVE PWTLQDTSYE MWLTPPKRCF AKQGNTVEVK
FDGCEDNVME YVVWTHIYLQ DNDSWVKVTS SVDAKGIYYT
CGQFKTYYVN FNKEAQKYGS TNHWEVCYGS TVICSPASVS
STVREVSIAE PTTYTPAQTT APTVSACTTE DGVSAPPRKR
ARGPSTNNTL CVANIRSVDS TINNIVTDNY NKHQRRNNCH
SAATPIVQLQ GDSNCLKCFR YRLNDKYKHL FELASSTWHW
ASPEAPHKNA IVTLTYSSEE QRQQFLNSVK IPPTIRHKVG
FMSLHLL

### 5. PLASMID: HPV104 (p7313me 6b/11e2 c/o mut)

Gene of Interest:

A fusion protein of 6be2 and 11e2 was constructed using 2×PCR with HPV102 and HPV110 as templates and appropriate designed primers. The fusion fragment 2.2 kb was cloned into p7313me expression vector with the 6be2 at the beginning of the fusion protein.

Cloning:

The 2.2 kb fusion was digested with Bam HI and Not I restriction enzymes and ligated into p7313me expression vector. Isolated clones were checked by sequencing and indicated no errors had been incorporated

6b/11e2 fusion sequence in HPV104

(Seq. ID No. 9)

ATGGAAGCTATTGCCAAGCGACTGGACGCCTGCCAGGAGCAGCTGCTGGA
GCTGTACGAGGAAAACAGCACAGACCTCCACAAGCACGTGCTGCACTGGA
AGTGCATGCGCCACGAGTCAGTGCTCCTGTACAAGGCCAAGCAGATGGGG
CTGTCCCACATCGGGATGCAGGTCGTGCCCCCGCTGAAGGTGAGCGAAGC
CAAGGGCCACAACGCTATCGAGATGCAGATGCACCTGGAGAGCCTGCTGC
GGACCGAATACAGCATGGAGCCCTGGACTCTCCAGGAGACGTCCTACGAA
ATGTGGCAGACTCCTCCGAAGCGCTGTTTCGCAAAGCGCGGCAAGACAGT
TGAGGTGAAATTCGATGGGTGCGCAAACAACACGATGGACTACGTGGTGT
GGACCGATGTCTACGTGCAGGACAATGACACCTGGGTGAAGGTACATAGT
ATGGTGGATGCCAAGGGCATCTATTACACCTGCGGGCAGTTCAAGACGTA
CTACGTCAACTTCGTCAAGGAAGCCGAAAAGTATGGTTCCACCAAGCACT
GGGAGGTGTGTTACGGGAGTACTGTGATCTGCAGCCCCGCCTCCGTGTCG
TCCACCACCCAGGAAGTGAGCATTCCGGAGAGCACCACATACACCCCGGC
CCAAACGAGCACGCTCGTCAGCAGCAGCACCAAGGAGGACGCCGTCCAGA
CGCCCCCCCGGAAGAGGGCCCGGGGGGTCCAGCAGTCTCCCTGCAATGCC
CTGTGCGTTGCTCACATCGGCCCTGTCGATTCTGGGAACCACAATCTCAT
CACGAACAACCACGACCAGCACCAAAGGCGCAACAACTCTAACAGCTCCG
CAACTCCAATAGTGCAGTTCCAGGGGGAGTCCAACTGCCTCAAGTGTTTC
CGCTACCGCCTCAACGACCGCCACCGCCACCTGTTCGACTTGATCAGTTC
CACGTGGCACTGGGCCAGCAGCAAGGCGCCCCACAAACACGCTATCGTGA
CGGTGACCTACGACTCCGAGGAGCAGAGGCAGCAGTTCCTGGACGTCGTG
AAGATTCCTCCGACAATCAGCCACAAGCTTGGCTTCATGTCCCTGCACCT
GCTGATGGAAGCCATCGCGAAGAGGCTCGACGCCTGCCAGGACCAGCTGC
TCGAGCTGTACGAGGAGAACAGCATTGACATCCATAAGCACATCATGCAC
TGGAAGTGCATTCGCCTGGAGAGCGTGCTGTTGCACAAGGCCAAGCAGAT
GGGCCTGTCCCACATAGGCCTTCAGGTGGTCCCCCCTCTGACCGTGTCAG
AGACAAAGGGCCATAACGCAATCGAGATGCAGATGCACCTCGAGTCGCTG
GCGAAAACACAGTACGGCGTGGAGCCATGGACCCTGCAGGACACCTCGTA
CGAAATGTGGCTGACCCCACCTAAGCGATGCTTCGCCAAACAGGGCAACA
CAGTGGAGGTGAAGTTCGACGGCTGTGAGGATAACGTTATGGAGTATGTC
GTGTGGACGCACATCTATCTGCAGGACAACGACAGTTGGGTGAAGGTGAC
CAGCTCCGTGGACGCGAAGGGCATCTACTATACCTGTGGGCAGTTTAAAA
CCTACTATGTGAACTTCAACAAAGAGGCCCAAAAGTATGGCTCCACCAAC
CACTGGGAGGTCTGCTATGGGAGCACGGTGATTTGCTCTCCCGCCAGCGT
GTCTAGCACTGTGCGCGAGGTGAGCATTGCCGAGCCGACCACGTACACCC
CTGCCCAGACGACCGCTCCGACCGTGTCTGCTTGTACTACCGAGGACGGC
GTGAGCGCTCCACCCAGGAAGCGTGCGAGGGGCCCAAGCACCAACAACAC
CCTCTGTGTGGCGAACATTCGCAGCGTCGACAGTACCATCAATAACATCG
TGACGGATAACTATAACAAGCACCAGAGGCGTAACAACTGTCACTCTGCC
GCAACCCCCATCGTGCAGCTCCAGGGAGACAGCAATTGCCTTAAGTGCTT
CCGCTATCGCCTCAACGACAAGTACAAGCACCTCTTTGAGCTCGCCTCGT
CGACGTGGCACTGGGCCTCACCCGAGGCACCTCACAAGAACGCCATCGTC
ACTCTCACTTACTCCAGTGAGGAGCAGAGACAGCAGTTTCTGAACAGCGT
GAAGATCCCACCGACGATCCGTCATAAGGTCGGCTTCATGTCACTGCATC
TCCTGA
Amino acid sequence

(Seq. ID No. 10)

MEAIAKRLDA CQEQLLELYE ENSTDLHKHV LHWKCMRHES
VLLYKAKQMG LSHIGMQVVP PLKVSEAKGH NAIEMQMHLE
SLLRTEYSME PWTLQETSYE MWQTPPKRCF AKRGKTVEVK
FDGCANNTMD YVVWTDVYVQ DNDTWVKVHS MVDAKGIYYT
CGQFKTYYVN FVKEAEKYGS TKHWEVCYGS TVICSPASVS
STTQEVSIPE STTYTPAQTS TLVSSSTKED AVQTPPRKRA
RGVQQSPCNA LCVAHIGPVD SGNHNLITNN HDQHQRRNNS
NSSATPIVQF QGESNCLKCF RYRLNDRHRH LFDLISSTWH
WASSKAPHKH AIVTVTYDSE EQRQQFLDVV KIPPTISHKL
GFMSLHLLME AIAKRLDACQ DQLLELYEEN SIDIHKHIMH
WKCIRLESVL LHKAKQMGLS HIGLQVVPPL TVSETKGHNA
IEMQMHLESL AKTQYGVEPW TLQDTSYEMW LTPPKRCFAK
QGNTVEVKFD GCEDNVMEYV VWTHIYLQDN DSWVKVTSSV
DAKGIYYTCG QFKTYYVNFN KEAQKYGSTN HWEVCYGSTV
ICSPASVSST VREVSIAEPT TYTPAQTTAP TVSACTTEDG
VSAPPRKRAR GPSTNNTLCV ANIRSVDSTI NNIVTDNYNK
HQRRNNCHSA ATPIVQLQGD SNCLKCFRYR LNDKYKHLFE
LASSTWHWAS PEAPHKNAIV TLTYSSEEQR QQFLNSVKIP
PTIRHKVGFM SLHLL

### 6. PLASMID: HPV105 (p7313me 11/6be2 c/o mut)

Gene of Interest:

A fusion protein of 6be2 and 11e2 was constructed using 2×PCR with HPV102 and HPV110 as templates and appropriate designed primers. The fusion fragment 2.2 kb was cloned into p7313me expression vector and with the 11e2 at the beginning of the fusion protein.

Cloning:

The 2.2 kb fusion was digested with Bam HI and Not I restriction enzymes and ligated into p7313me expression vector. Isolated clones were checked by sequencing and indicated no errors had been incorporated.

11/6bE2 fusion sequence in HPV105

(Seq. ID No. 11)

ATGGAAGCCATCGCGAAGAGGCTCGACGCCTGCCAGGACCAGCTGCTCGA
GCTGTACGAGGAGAACAGCATTGACATCCATAAGCACATCATGCACTGGA
AGTGCATTCGCCTGGAGAGCGTGCTGTTGCACAAGGCCAAGCAGATGGGC
CTGTCCCACATAGGCCTTCAGGTGGTCCCCCCTCTGACCGTGTCAGAGAC
AAAGGGCCATAACGCAATCGAGATGCAGATGCACCTCGAGTCGCTGGCGA
AAACACAGTACGGCGTGGAGCCATGGACCCTGCAGGACACCTCGTACGAA
ATGTGGCTGACCCCACCTAAGCGATGCTTCGCCAAACAGGGCAACACAGT
GGAGGTGAAGTTCGACGGCTGTGAGGATAACGTTATGGAGTATGTCGTGT
GGACGCACATCTATCTGCAGGACAACGACAGTTGGGTGAAGGTGACCAGC
TCCGTGGACGCGAAGGGCATCTACTATACCTGTGGGCAGTTTAAAACCTA
CTATGTGAACTTCAACAAAGAGGCCCAAAAGTATGGCTCCACCAACCACT
GGGAGGTCTGCTATGGGAGCACGGTGATTTGCTCTCCCGCCAGCGTGTCT
AGCACTGTGCGCGAGGTGAGCATTGCCGAGCCGACCACGTACACCCCTGC
CCAGACGACCGCTCCGACCGTGTCTGCTTGTACTACCGAGGACGGCGTGA
GCGCTCCACCCAGGAAGCGTGCGAGGGGCCCAAGCACCAACAACACCCTC
TGTGTGGCGAACATTCGCAGCGTCGACAGTACCATCAATAACATCGTGAC
GGATAACTATAACAAGCACCAGAGGCGTAACAACTGTCACTCTGCCGCAA
CCCCCATCGTGCAGCTCCAGGGAGACAGCAATTGCCTTAAGTGCTTCCGC
TATCGCCTCAACGACAAGTACAAGCACCTCTTTGAGCTCGCCTCGTCGAC
GTGGCACTGGGCCTCACCCGAGGCACCTCACAAGAACGCCATCGTCACTC
TCACTTACTCCAGTGAGGAGCAGAGACAGCAGTTTCTGAACAGCGTGAAG
ATCCCACCGACGATCCGTCATAAGGTCGGCTTCATGTCACTGCATCTCCT
GATGGAAGCTATTGCCAAGCGACTGGACGCCTGCCAGGAGCAGCTGCTGG
AGCTGTACGAGGAAAACAGCACAGACCTCCACAAGCACGTGCTGCACTGG
AAGTGCATGCGCCACGAGTCAGTGCTCCTGTACAAGGCCAAGCAGATGGG
GCTGTCCCACATCGGGATGCAGGTCGTGCCCCCGCTGAAGGTGAGCGAAG
CCAAGGGCCACAACGCTATCGAGATGCAGATGCACCTGGAGAGCCTGCTG
CGGACCGAATACAGCATGGAGCCCTGGACTCTCCAGGAGACGTCCTACGA
AATGTGGCAGACTCCTCCGAAGCGCTGTTTCGCAAAGCGCGGCAAGACAG
TTGAGGTGAAATTCGATGGGTGCGCAAACAACACGATGGACTACGTGGTG
TGGACCGATGTCTACGTGCAGGACAATGACACCTGGGTGAAGGTACATAG
TATGGTGGATGCCAAGGGCATCTATTACACCTGCGGGCAGTTCAAGACGT
ACTACGTCAACTTCGTCAAGGAAGCCGAAAAGTATGGTTCCACCAAGCAC
TGGGAGGTGTGTTACGGGAGTACTGTGATCTGCAGCCCCGCCTCCGTGTC
GTCCACCACCCAGGAAGTGAGCATTCCGGAGAGCACCACATACACCCCGG
CCCAAACGAGCACGCTCGTCAGCAGCAGCACCAAGGAGGACGCCGTCCAG
ACGCCCCCCCGGAAGAGGGCCCGGGGGGTCCAGCAGTCTCCCTGCAATGC
CCTGTGCGTTGCTCACATCGGCCCTGTCGATTCTGGGAACCACAATCTCA
TCACGAACAACCACGACCAGCACCAAAGGCGCAACAACTCTAACAGCTCC
GCAACTCCAATAGTGCAGTTCCAGGGGGAGTCCAACTGCCTCAAGTGTTT
CCGCTACCGCCTCAACGACCGCCACCGCCACCTGTTCGACTTGATCAGTT
CCACGTGGCACTGGGCCAGCAGCAAGGCGCCCCACAAACACGCTATCGTG
ACGGTGACCTACGACTCCGAGGAGCAGAGGCAGCAGTTCCTGGACGTCGT
GAAGATTCCTCCGACAATCAGCCACAAGCTTGGCTTCATGTCCCTGCACC
TGCTGA
Amino acid sequence

(Seq. ID No. 12)

MEAIAKRLDA CQDQLLELYE ENSIDIHKHI MHWKCIRLES
VLLHKAKQMG LSHIGLQVVP PLTVSETKGH NAIEMQMHLE
SLAKTQYGVE PWTLQDTSYE MWLTPPKRCF AKQGNTVEVK
FDGCEDNVME YVVWTHIYLQ DNDSWVKVTS SVDAKGIYYT
CGQFKTYYVN FNKEAQKYGS TNHWEVCYGS TVICSPASVS
STVREVSIAE PTTYTPAQTT APTVSACTTE DGVSAPPRKR
ARGPSTNNTL CVANIRSVDS TTNNIVTDNY NKHQRRNNCH
SAATPIVQLQ GDSNCLKCFR YRLNDKYKHL FELASSTWHW
ASPEAPHKNA IVTLTYSSEE QRQQFLNSVK IPPTIRHKVG
FMSLHLLMEA IAKRLDACQE QLLELYEENS TDLHKHVLHW
KCMRHESVLL YKAKQMGLSH IGMQVVPPLK VSEAKGHNAI
EMQMHLESLL RTEYSMEPWT LQETSYEMWQ TPPKRCFAKR
GKTVEVKFDG CANNTMDYVV WTDVYVQDND TWVKVHSMVD
AKGIYYTCGQ FKTYYVNFVK EAEKYGSTKH WEVCYGSTVI
CSPASVSSTT QEVSIPESTT YTPAQTSTLV SSSTKEDAVQ
TPPRKRARGV QQSPCNALCV AHIGPVDSGN HNLITNNHDQ
HQRRNNSNSS ATPIVQFQGE SNCLKCFRYR LNDRHRHLFD
LISSTWHWAS SKAPHKHAIV TVTYDSEEQR QQFLDVVKIP
PTISHKLGFM SLHLL

### 7. PLASMID: HPV108 (p7313ie 6be1 c/o mut)

Gene of Interest:

Codon optimised mutated 6be1 was transferred from p7313p1c 6be1 c/o mut clone N into vector p7313ie.

Cloning:

The 6be1 c/o mut fragment was cut out of the p7313p1c 6be2 clone by Not I and Bam HI restriction digests. This fragment was then ligated into p7313ie vector using these sites. The gene is under the control of the ie promoter (immediate early cmv+exon1) and followed by a rabbit b-globin poly-adenylation signal.

6be1 sequence in p7313ie

(Seq. ID No. 13)

ATGGCAGACGATTCCGGTACTGAGAACGAAGGTTCTGGTTGTACCGGTTG
GTTCATGGTTGAAGCAATCGTTCAGCATCCGACTGGTACCCAGATCTCCG
ATGACGAAGACGAAGAAGTTGAAGATTCTGGTTACGACATGGTTGACTTC
ATCGATGACTCCAACATCACTCATAACTCTCTGGAAGCACAGGCTCTGTT
TAACCGCCAGGAAGCTGATACCCATTACGCTACTGTTCAGGACCTGGGAG
GCAAATATCTGGGCTCTCCGTACGTTTCCCCGATCAACACTATCGCAGAA
GCAGTTGAGTCTGAAATCTCCCCGCGCCTGGACGCTATCAAACTGACTCG
TCAGCCGAAGAAGGTTAAACGTCGTCTGTTCCAGACTCGTGAACTGACCG
ACTCCGGTTACGGTTATAGCGAAGTTGAGGCTGGCACCGGCACCCAGGTT
GAAAAACACGGTGTACCGGAAAACGGCGGCGACGGTCAGGAAAAGGACAC
CGGCCGCGACATCGAGGGTGAGGAACACACCGAAGCTGAAGCTCCGACTA
ACTCTGTTCGTGAACACGCAGGTACTGCGGGTATCCTGGAACTGCTGAAA
TGCAAAGACCTGCGCGCGGCTCTGCTGGGCAAATTCAAAGAATGCTTCGG
CCTGTCTTTCATTGACCTGATCCGTCCGTTTAAGTCTGACAAAACTACCT
GTCTGGACTGGGTTGTAGCAGGCTTCGGCATCCACCACTCTATCTCTGAA
GCATTCCAGAAACTGATCGAGCCGCTGTCTCTGTACGCGCACATCCAGTG
GCTGACTAACGCTTGGGGTATGGTTCTGCTGGTACTGCTGCGCTTTAAAG
TAAACAAATCTCGTTCCACTGTTGCTCGTACTCTGGCTACCCTGCTGAAC
ATCCCGGAGAACCAGATGCTGATCGAACCGCCGAAAATCCAGTCTGGTGT
AGCTGCACTGTACTGGTTTCGTACTGGCATCTCTAACGCTAGCACTGTTA
TCGGTGAAGCACCGGAATGGATCACTCGTCAGACCGTTATCGAACACGGT
CTGGCAGATTCTCAGTTCAAACTGACTGAAATGGTTCAGTGGGCATACGA
CAACGACATCTGCGAGGAATCTGAAATTGCGTTCGAATACGCTCAGCGTG
GCGACTTCGACTCCAACGCTCGTGCTTTCCTGAACAGCAACATGCAGGCT
AAATACGTAAAAGACTGCGCTACCATGTGCCGTCACTACAAACACGCGGA
AATGCGTAAAATGTCTATCAAACAGTGGATCAAGCACCGCGGTTCTAAAA
TCGAAGGTACCGGTAACTGGAAACCGATCGTTCAGTTCCTGCGCCATCAG
AACATCGAATTCATCCCGTTCCTGACCAAATTCAAGCTGTGGCTGCACGG
TACCCCGAAAAAAAACTGCATCGCTATCGTAGGTCCACCGGACACTGACA
AGTCTTACTTCTGTATGTCCCTGATCTCTTTCCTGGGCGGCACTGTAATC
TCTCACGTTAACTCTTCCTCCCATTTCTGGCTGCAGCCACTGGTAGACGC
GAAAGTAGCTCTGCTGGACGACGCGACCCAGCCGTGCTGGATCTACATGG
ATACTTACATGCGCAACCTGCTGGACGGTAACCCGATGTCTATCGACCGT
AAACACAAAGCGCTGACTCTGATCAAGTGCCCGCCGCTGCTGGTAACTTC
TAACATCGACATCACCAAGGAAGATAAATACAAGTACCTGCATACCCGTG
TTACTACCTTTACTTTCCCGAACCCGTTCCCGTTTGATCGTAACGGTAAC
GCTGTTTACGAACTGTCCAACACTAACTGGAAATGCTTCTTCGAGCGTCT
GTCTTCCTCCCTGGACATCCAGGACTCTGAAGATGAAGAAGATGGTTCTA
ACTCTCAGGCTTTCCGTTGTGTTCCGGGTACTGTTGTTCGTACTCTGTGA
Amino acid sequence

(Seq. ID No. 14)

MADDSGTENE GSGCTGWFMV EAIVQHPTGT QISDDEDEEV
EDSGYDMVDF IDDSNITHNS LEAQALFNRQ EADTHYATVQ
DLGGKYLGSP YVSPTNTIAE AVESEISPRL DAIKLTRQPK
KVKRRLFQTR ELTDSGYGYS EVEAGTGTQV EKHGVPENGG
DGQEKDTGRD IEGEEHTEAE APTNSVREHA GTAGILELLK
CKDLRAALLG KFKECFGLSF IDLIRPFKSD KTTCLDWVVA
GFGIHHSISE AFQKLIEPLS LYAHIQWLTN AWGMVLLVLL
REKVNKSRST VARTLATLLN IPENQMLIEP PKIQSGVAAL
YWFRTGISNA STVIGEAPEW ITRQTVIEHG LADSQFKLTE
MVQWAYDNDI CEESEIAFEY AQRGDFDSNA RAFLNSNMQA
KYVKDCATMC RHYKHAEMRK MSIKQWIKHR GSKIEGTGNW
KPIVQFLRHQ NIEFIPFLTK FKLWLHGTPK KNCIAIVGPP
DTDKSYFCMS LISFLGGTVI SHVNSSSHFW LQPLVDAKVA
LLDDATQPCW IYMDTYMRNL LDGNPMSIDR KHKALTLIKC
PPLLVTSNID ITKEDKYKYL HTRVTTFTFP NPFPFDRNGN
AVYELSNTNW KCFFERLSSS LDIQDSEDEE DGSNSQAFRC
VPGTVVRTL

### 8. PLASMID: HPV110 (p7313ie 6be2 c/o mut)

Gene of Interest:

Codon optimised mutated 6be2 was transferred from pWRG7077 6be2 into vector p7313ie.

Cloning:

The 6be2 c/o mut fragment was cut out of pWRG7077 6be2 clone by Not I and Bam HI restriction digests. This fragment was then ligated into p7313ie vector using these sites. The gene is under the control of the ie promoter (immediate early cmv+exon1) and followed by a rabbit b-globin poly-adenylation signal.

6be2 sequence in p7313ie

(Seq. ID No. 15)

ATGGAAGCTATTGCCAAGCGACTGGACGCCTGCCAGGAGCAGCTGCTGGA
GCTGTACGAGGAAAACAGCACAGACCTCCACAAGCACGTGCTGCACTGGA
AGTGCATGCGCCACGAGTCAGTGCTCCTGTACAAGGCCAAGCAGATGGGG
CTGTCCCACATCGGGATGCAGGTCGTGCCCCCGCTGAAGGTGAGCGAAGC
CAAGGGCCACAACGCTATCGAGATGCAGATGCACCTGGAGAGCCTGCTGC
GGACCGAATACAGCATGGAGCCCTGGACTCTCCAGGAGACGTCCTACGAA
ATGTGGCAGACTCCTCCGAAGCGCTGTTTCGCAAAGCGCGGCAAGACAGT
TGAGGTGAAATTCGATGGGTGCGCAAACAACACGATGGACTACGTGGTGT
GGACCGATGTCTACGTGCAGGACAATGACACCTGGGTGAAGGTACATAGT
ATGGTGGATGCCAAGGGCATCTATTACACCTGCGGGCAGTTCAAGACGTA
CTACGTCAACTTCGTCAAGGAAGCCGAAAAGTATGGTTCCACCAAGCACT
GGGAGGTGTGTTACGGGAGTACTGTGATCTGCAGCCCCGCCTCCGTGTCG
TCCACCACCCAGGAAGTGAGCATTCCGGAGAGCACCACATACACCCCGGC
CCAAACGAGCACGCTCGTCAGCAGCAGCACCAAGGAGGACGCCGTCCAGA
CGCCCCCCCGGAAGAGGGCCCGGGGGGTCCAGCAGTCTCCCTGCAATGCC
CTGTGCGTTGCTCACATCGGCCCTGTCGATTCTGGGAACCACAATCTCAT
CACGAACAACCACGACCAGCACCAAAGGCGCAACAACTCTAACAGCTCCG
CAACTCCAATAGTGCAGTTCCAGGGGGAGTCCAACTGCCTCAAGTGTTTC
CGCTACCGCCTCAACGACCGCCACCGCCACCTGTTCGACTTGATCAGTTC
CACGTGGCACTGGGCCAGCAGCAAGGCGCCCCACAAACACGCTATCGTGA
CGGTGACCTACGACTCCGAGGAGCAGAGGCAGCAGTTCCTGGACGTCGTG
AAGATTCCTCCGACAATCAGCCACAAGCTTGGCTTCATGTCCCTGCACCT
GCTGTGA
Amino acid sequence

(Seq. ID No. 16)

MEAIAKRLDA CQEQLLELYE ENSTDLHKHV LHWKCMRHES
VLLYKAKQMG LSHIGMQVVP PLKVSEAKGH NAIEMQMHLE
SLLRTEYSME PWTLQETSYE MWQTPPKRCF AKRGKTVEVK
FDGCANNTMD YVVWTDVYVQ DNDTWVKVHS MVDAKGIYYT
CGQFKTYYVN FVKEAEKYGS TKHWEVCYGS TVICSPASVS
STTQEVSIPE STTYTPAQTS TLVSSSTKED AVQTPPRKRA
RGVQQSPCNA LCVAHIGPVD SGNHNLITNN HDQHQRRNNS
NSSATPIVQF QGESNCLKCF RYRLNDRHRH LFDLISSTWH
WASSKAPHKH AIVTVTYDSE EQRQQFLDVV KIPPTISHKL
GFMSLHLL

### 9. PLASMID: HPV116 (p7313ie 6be1.6be2.11e2)

Gene of Interest:

The gene for the polyprotein in construct HPV116 is a triple fusion protein comprised in order of 6be1, 6be2, 11e2 all codon optimised and mutated. The polyprotein gene was assembled by PCR from using 2 previous PCR fragments; 6be1 and 6b/11e2. The size of the gene is ˜4.1 kb, producing a polyprotein of ˜70 kD, observed by PAGE and Western blot.

Cloning:

The polyprotein gene was digested with Bam HI+Not I restriction enzymes and ligated into p7313ie vector. Sequencing analysis of selected clones had indicated the ‘odd’ base change, but this was overcome by various fragment swapping. A resulting clone hpv116 #1 was found to have no errors.

Polyprotein sequence in HPV116

(Seq. ID No. 17)

ATGGCAGACGATTCCGGTACTGAGAACGAAGGTTCTGGTTGTACCGGTTG
GTTCATGGTTGAAGCAATCGTTCAGCATCCGACTGGTACCCAGATCTCCG
ATGACGAAGACGAAGAAGTTGAAGATTCTGGTTACGACATGGTTGACTTC
ATCGATGACTCCAACATCACTCATAACTCTCTGGAAGCACAGGCTCTGTT
TAACCGCCAGGAAGCTGATACCCATTACGCTACTGTTCAGGACCTGGGAG
GCAAATATCTGGGCTCTCCGTACGTTTCCCCGATCAACACTATCGCAGAA
GCAGTTGAGTCTGAAATCTCCCCGCGCCTGGACGCTATCAAACTGACTCG
TCAGCCGAAGAAGGTTAAACGTCGTCTGTTCCAGACTCGTGAACTGACCG
ACTCCGGTTACGGTTATAGCGAAGTTGAGGCTGGCACCGGCACCCAGGTT
GAAAAACACGGTGTACCGGAAAACGGCGGCGACGGTCAGGAAAAGGACAC
CGGCCGCGACATCGAGGGTGAGGAACACACCGAAGCTGAAGCTCCGACTA
ACTCTGTTCGTGAACACGCAGGTACTGCGGGTATCCTGGAACTGCTGAAA
TGCAAAGACCTGCGCGCGGCTCTGCTGGGCAAATTCAAAGAATGCTTCGG
CCTGTCTTTCATTGACCTGATCCGTCCGTTTAAGTCTGACAAAACTACCT
GTCTGGACTGGGTTGTAGCAGGCTTCGGCATCCACCACTCTATCTCTGAA
GCATTCCAGAAACTGATCGAGCCGCTGTCTCTGTACGCGCACATCCAGTG
GCTGACTAACGCTTGGGGTATGGTTCTGCTGGTACTGCTGCGCTTTAAAG
TAAACAAATCTCGTTCCACTGTTGCTCGTACTCTGGCTACCCTGCTGAAC
ATCCCGGAGAACCAGATGCTGATCGAACCGCCGAAAATCCAGTCTGGTGT
AGCTGCACTGTACTGGTTTCGTACTGGCATCTCTAACGCTAGCACTGTTA
TCGGTGAAGCACCGGAATGGATCACTCGTCAGACCGTTATCGAACACGGT
CTGGCAGATTCTCAGTTCAAACTGACTGAAATGGTTCAGTGGGCATACGA
CAACGACATCTGCGAGGAATCTGAAATTGCGTTCGAATACGCTCAGCGTG
GCGACTTCGACTCCAACGCTCGTGCTTTCCTGAACAGCAACATGCAGGCT
AAATACGTAAAAGACTGCGCTACCATGTGCCGTCACTACAAACACGCGGA
AATGCGTAAAATGTCTATCAAACAGTGGATCAAGCACCGCGGTTCTAAAA
TCGAAGGTACCGGTAACTGGAAACCGATCGTTCAGTTCCTGCGCCATCAG
AACATCGAATTCATCCCGTTCCTGACCAAATTCAAGCTGTGGCTGCACGG
TACCCCGAAAAAAAACTGCATCGCTATCGTAGGTCCACCGGACACTGACA
AGTCTTACTTCTGTATGTCCCTGATCTCTTTCCTGGGCGGCACTGTAATC
TCTCACGTTAACTCTTCCTCCCATTTCTGGCTGCAGCCACTGGTAGACGC
GAAAGTAGCTCTGCTGGACGACGCGACCCAGCCGTGCTGGATCTACATGG
ATACTTACATGCGCAACCTGCTGGACGGTAACCCGATGTCTATCGACCGT
AAACACAAAGCGCTGACTCTGATCAAGTGCCCGCCGCTGCTGGTAACTTC
TAACATCGACATCACCAAGGAAGATAAATACAAGTACCTGCATACCCGTG
TTACTACCTTTACTTTCCCGAACCCGTTCCCGTTTGATCGTAACGGTAAC
GCTGTTTACGAACTGTCCAACACTAACTGGAAATGCTTCTTCGAGCGTCT
GTCTTCCTCCCTGGACATCCAGGACTCTGAAGATGAAGAAGATGGTTCTA
ACTCTCAGGCTTTCCGTTGTGTTCCGGGTACTGTTGTTCGTACTCTGATG
GAAGCTATTGCCAAGCGACTGGACGCCTGCCAGGAGCAGCTGCTGGAGCT
GTACGAGGAAAACAGCACAGACCTCCACAAGCACGTGCTGCACTGGAAGT
GCATGCGCCACGAGTCAGTGCTCCTGTACAAGGCCAAGCAGATGGGGCTG
TCCCACATCGGGATGCAGGTCGTGCCCCCGCTGAAGGTGAGCGAAGCCAA
GGGCCACAACGCTATCGAGATGCAGATGCACCTGGAGAGCCTGCTGCGGA
CCGAATACAGCATGGAGCCCTGGACTCTCCAGGAGACGTCCTACGAAATG
TGGCAGACTCCTCCGAAGCGCTGTTTCGCAAAGCGCGGCAAGACAGTTGA
GGTGAAATTCGATGGGTGCGCAAACAACACGATGGACTACGTGGTGTGGA
CCGATGTCTACGTGCAGGACAATGACACCTGGGTGAAGGTACATAGTATG
GTGGATGCCAAGGGCATCTATTACACCTGCGGGCAGTTCAAGACGTACTA
CGTCAACTTCGTCAAGGAAGCCGAAAAGTATGGTTCCACCAAGCACTGGG
AGGTGTGTTACGGGAGTACTGTGATCTGCAGCCCCGCCTCCGTGTCGTCC
ACCACCCAGGAAGTGAGCATTCCGGAGAGACCACATACACCCCGGCCCAA
ACGAGCACGCTCGTCAGCAGCAGCACCAAGGAGGACGCCGTCCAGACGCC
CCCCCGGAAGAGGGCCCGGGGGGTCCAGCAGTCTCCCTGCAATGCCCTGT
GCGTTGCTCACATCGGCCCTGTCGATTCTGGGAACCACAATCTCATCACG
AACAACCACGACCAGCACCAAAGGCGCAACAACTCTAACAGCTCCGCAAC
TCCAATAGTGCAGTTCCAGGGGGAGTCCAACTGCCTCAAGTGTTTCCGCT
ACCGCCTCAACGACCGCCACCGCCACCTGTTCGACTTGATCAGTTCCACG
TGGCACTGGGCCAGCAGCAAGGCGCCCCACAAACACGCTATCGTGACGGT
GACCTACGACTCCGAGGAGCAGAGGCAGCAGTTCCTGGACGTCGTGAAGA
TTCCTCCGACAATCAGCCACAAGCTTGGCTTCATGTCCCTGCACCTGCTG
ATGGAAGCCATCGCGAAGAGGCTCGACGCCTGCCAGGACCAGCTGCTCGA
GCTGTACGAGGAGAACAGCATTGACATCCATAAGCACATCATGCACTGGA
AGTGCATTCGCCTGGAGAGCGTGCTGTTGCACAAGGCCAAGCAGATGGGC
CTGTCCCACATAGGCCTTCAGGTGGTCCCCCCTCTGACCGTGTCAGAGAC
AAAGGGCCATAACGCAATCGAGATGCAGATGCACCTCGAGTCGCTGGCGA
AAACACAGTACGGCGTGGAGCCATGGACCCTGCAGGACACCTCGTACGAA
ATGTGGCTGACCCCACCTAAGCGATGCTTCGCCAAACAGGGCAACACAGT
GGAGGTGAAGTTCGACGGCTGTGAGGATAACGTTATGGAGTATGTCGTGT
GGACGCACATCTATCTGCAGGACAACGACAGTTGGGTGAAGGTGACCAGC
TCCGTGGACGCGAAGGGCATCTACTATACCTGTGGGCAGTTTAAAACCTA
CTATGTGAACTTCAACAAAGAGGCCCAAAAGTATGGCTCCACCAACCACT
GGGAGGTCTGCTATGGGAGCACGGTGATTTGCTCTCCCGCCAGCGTGTCT
AGCACTGTGCGCGAGGTGAGCATTGCCGAGCCGACCACGTACACCCCTGC
CCAGACGACCGCTCCGACCGTGTCTGCTTGTACTACCGAGGACGGCGTGA
GCGCTCCACCCAGGAAGCGTGCGAGGGGCCCAAGCACCAACAACACCCTC
TGTGTGGCGAACATTCGCAGCGTCGACAGTACCATCAATAACATCGTGAC
GGATAACTATAACAAGCACCAGAGGCGTAACAACTGTCACTCTGCCGCAA
CCCCCATCGTGCAGCTCCAGGGAGACAGCAATTGCCTTAAGTGCTTCCGC
TATCGCCTCAACGACAAGTACAAGCACCTCTTTGAGCTCGCCTCGTCGAC
GTGGCACTGGGCCTCACCCGAGGCACCTCACAAGAACGCCATCGTCACTC
TCACTTACTCCAGTGAGGAGCAGAGACAGCAGTTTCTGAACAGCGTGAAG
ATCCCACCGACGATCCGTCATAAGGTCGGCTTCATGTCACTGCATCTCCT
GTGA
Amino acid sequence

(Seq. ID No. 18)

MADDSGTENE GSGCTGWFMV EAIVQHPTGT QISDDEDEEV
EDSGYDMVDF IDDSNITHNS LEAQALFNRQ EADTHYATVQ
DLGGKYLGSP YVSPINTIAE AVESEISPRL DAIKLTRQPK
KVKRRLFQTR ELTDSGYGYS EVEAGTGTQV EKHGVPENGG
DGQEKDTGRD IEGEEHTEAE APTNSVREHA GTAGILELLK
CKDLRAALLG KFKECFGLSF IDLIRPFKSD KTTCLDWVVA
GFGIHHSISE AFQKLIEPLS LYAHIQWLTN AWGMVLLVLL
RFKVNKSRST VARTLATLLN IPENQMLIEP PKIQSGVAAL
YWFRTGISNA STVIGEAPEW ITRQTVIEHG LADSQFKLTE
MVQWAYDNDI CEESEIAFEY AQRGDFDSNA RAFLNSNMQA
KYVKDCATMC RHYKHAEMRK MSIKQWIKHR GSKIEGTGNW
KPIVQFLRHQ NIEFIPFLTK FKLWLHGTPK KNCIAIVGPP
DTDKSYFCMS LISFLGGTVI SHVNSSSHFW LQPLVDAKVA
LLDDATQPCW IYMDTYMRNL LDGNPMSIDR KHKALTLIKC
PPLLVTSNID ITKEDKYKYL HTRVTTFTFP NPFPFDRNGN
AVYELSNTNW KCFFERLSSS LDIQDSEDEE DGSNSQAFRC
VPGTVVRTLM EAIAKRLDAC QEQLLELYEE NSTDLHKHVL
HWKCMRHESV LLYKAKQMGL SHIGMQVVPP LKVSEAKGHN
AIEMQMHLES LLRTEYSMEP WTLQETSYEM WQTPPKRCFA
KRGKTVEVKF DGCANNTMDY VVWTDVYVQD NDTWVKVHSM
VDAKGIYYTC GQFKTYYVNF VKEAEKYGST KHWEVCYGST
VICSPASVSS TTQEVSIPES TTYTPAQTST LVSSSTKEDA
VQTPPRKRAR GVQQSPCNAL CVAHIGPVDS GNHNLITNNH
DQHQRRNNSN SSATPIVQFQ GESNCLKCFR YRLNDRHRHL
FDLISSTWHW ASSKAPHKHA IVTVTYDSEE QRQQFLDVVK
IPPTISHKLG FMSLHLLMEA IAKRLDACQD QLLELYEENS
IDIHKHIMHW KCIRLESVLL HKAKQMGLSH IGLQVVPPLT
VSETKGHNAI EMQMHLESLA KTQYGVEPWT LQDTSYEMWL
TPPKRCFAKQ GNTVEVKFDG CEDNVMEYVV WTHIYLQDND
SWVKVTSSVD AKGIYYTCGQ FKTYYVNFNK EAQKYGSTNH
WEVCYGSTVI CSPASVSSTV REVSIAEPTT YTPAQTTAPT
VSACTTEDGV SAPPRKRARG PSTMNTLCVA NIRSVDSTTN
NIVTDNYNKH QRRNNCHSAA TPIVQLQGDS NCLKCFRYRL
NDKYKHLFEL ASSTWHWASP EAPHKNAIVT LTYSSEEQRQ
QFLNSVKIPP TIRHKVGFMS LHLL

### 10. PLASMID: HPV117 (p7313ie 6be2.6be1.11e2)

Gene of Interest:

The gene for the polyprotein in construct HPV117 is a triple fusion protein comprised in order of 6be2, 6be1, 11e2 all codon optimised and mutated. The polyprotein gene was assembled by PCR from using 3 previous PCR fragments; 6be1 and 6be2 and 11e2. The size of the gene is 4.1 kb, producing a polyprotein of 170 kD, observed by PAGE and Western blot.

Cloning:

The polyprotein gene was digested with Bam HI+Not I restriction enzymes and ligated into p7313ie vector. Sequencing analysis of selected clones had indicated the ‘odd’ base change, but this was overcome by various fragment swapping. A resulting clone hpv117 #6 was found to have no errors.

Polyprotein sequence in HPV117 (Seq. ID No. 19)
ATGGAAGCTATTGCCAAGCGACTGGACGCCTGCCAGGAGCAGCTGCTGGA
GCTGTACGAGGAAAACAGCACAGACCTCCACAAGCACGTGCTGCACTGGA
AGTGCATGCGCCACGAGTCAGTGCTCCTGTACAAGGCCAAGCAGATGGGG
CTGTCCCACATCGGGATGCAGGTCGTGCCCCCGCTGAAGGTGAGCGAAGC
CAAGGGCCACAACGCTATCGAGATGCAGATGCACCTGGAGAGCCTGCTGC
GGACCGAATACAGCATGGAGCCCTGGACTCTCCAGGAGACGTCCTACGAA
ATGTGGCAGACTCCTCCGAAGCGCTGTTTCGCAAAGCGCGGCAAGACAGT
TGAGGTGAAATTCGATGGGTGCGCAAACAACACGATGGACTACGTGGTGT
GGACCGATGTCTACGTGCAGGACAATGACACCTGGGTGAAGGTACATAGT
ATGGTGGATGCCAAGGGCATCTATTACACCTGCGGGCAGTTCAAGACGTA
CTACGTCAACTTCGTCAAGGAAGCCGAAAAGTATGGTTCCACCAAGCACT
GGGAGGTGTGTTACGGGAGTACTGTGATCTGCAGCCCCGCCTCCGTGTCG
TCCACCACCCAGGAAGTGAGCATTCCGGAGAGCACCACATACACCCCGGC
CCAAACGAGCACGCTCGTCAGCAGCAGCACCAAGGAGGACGCCGTCCAGA
CGCCCCCCCGGAAGAGGGCCCGGGGGGTCCAGCAGTCTCCCTGCAATGCC
CTGTGCGTTGCTCACATCGGCCCTGTCGATTCTGGGAACCACAATCTCAT
CACGAACAACCACGACCAGCACCAAAGGCGCAACAACTCTAACAGCTCCG
CAACTCCAATAGTGCAGTTCCAGGGGGAGTCCAACTGCCTCAAGTGTTTC
CGCTACCGCCTCAACGACCGCCACCGCCACCTGTTCGACTTGATCAGTTC
CACGTGGCACTGGGCCAGCAGCAAGGCGCCCCACAAACACGCTATCGTGA
CGGTGACCTACGACTCCGAGGAGCAGAGGCAGCAGTTCCTGGACGTCGTG
AAGATTCCTCCGACAATCAGCCACAAGCTTGGCTTCATGTCCCTGCACCT
GCTGATGGCAGACGATTCCGGTACTGAGAACGAAGGTTCTGGTTGTACCG
GTTGGTTCATGGTTGAAGCAATCGTTCAGCATCCGACTGGTACCCAGATC
TCCGATGACGAAGACGAAGAAGTTGAAGATTCTGGTTACGACATGGTTGA
CTTCATCGATGACTCCAACATCACTCATAACTCTCTGGAAGCACAGGCTC
TGTTTAACCGCCAGGAAGCTGATACCCATTACGCTACTGTTCAGGACCTG
GGAGGCAAATATCTGGGCTCTCCGTACGTTTCCCCGATCAACACTATCGC
AGAAGCAGTTGAGTCTGAAATCTCCCCGCGCCTGGACGCTATCAAACTGA
CTCGTCAGCCGAAGAAGGTTAAACGTCGTCTGTTCCAGACTCGTGAACTG
ACCGACTCCGGTTACGGTTATAGCGAAGTTGAGGCTGGCACCGGCACCCA
GGTTGAAAAACACGGTGTACCGGAAAACGGCGGCGACGGTCAGGAAAAGG
ACACCGGCCGCGACATCGAGGGTGAGGAACACACCGAAGCTGAAGCTCCG
ACTAACTCTGTTCGTGAACACGCAGGTACTGCGGGTATCCTGGAACTGCT
GAAATGCAAAGACCTGCGCGCGGCTCTGCTGGGCAAATTCAAAGAATGCT
TCGGCCTGTCTTTCATTGACCTGATCCGTCCGTTTAAGTCTGACAAAACT
ACCTGTCTGGACTGGGTTGTAGCAGGCTTCGGCATCCACCACTCTATCTC
TGAAGCATTCCAGAAACTGATCGAGCCGCTGTCTCTGTACGCGCACATCC
AGTGGCTGACTAACGCTTGGGGTATGGTTCTGCTGGTACTGCTGCGCTTT
AAAGTAAACAAATCTCGTTCCACTGTTGCTCGTACTCTGGCTACCCTGCT
GAACATCCCGGAGAACCAGATGCTGATCGAACCGCCGAAAATCCAGTCTG
GTGTAGCTGCACTGTACTGGTTTCGTACTGGCATCTCTAACGCTAGCACT
GTTATCGGTGAAGCACCGGAATGGATCACTCGTCAGACCGTTATCGAACA
CGGTCTGGCAGATTCTCAGTTCAAACTGACTGAAATGGTTCAGTGGGCAT
ACGACAACGACATCTGCGAGGAATCTGAAATTGCGTTCGAATACGCTCAG
CGTGGCGACTTCGACTCCAACGCTCGTGCTTTCCTGAACAGCAACATGCA
GGCTAAATACGTAAAAGACTGCGCTACCATGTGCCGTCACTACAAACACG
CGGAAATGCGTAAAATGTCTATCAAACAGTGGATCAAGCACCGCGGTTCT
AAAATCGAAGGTACCGGTAACTGGAAACCGATCGTTCAGTTCCTGCGCCA
TCAGAACATCGAATTCATCCCGTTCCTGACCAAATTCAAGCTGTGGCTGC
ACGGTACCCCGAAAAAAAACTGCATCGCTATCGTAGGTCCACCGGACACT
GACAAGTCTTACTTCTGTATGTCCCTGATCTCTTTCCTGGGCGGCACTGT
AATCTCTCACGTTAACTCTTCCTCCCATTTCTGGCTGCAGCCACTGGTAG
ACGCGAAAGTAGCTCTGCTGGACGACGCGACCCAGCCGTGCTGGATCTAC
ATGGATACTTACATGCGCAACCTGCTGGACGGTAACCCGATGTCTATCGA
CCGTAAACACAAAGCGCTGACTCTGATCAAGTGCCCGCCGCTGCTGGTAA
CTTCTAACATCGACATCACCAAGGAAGATAAATACAAGTACCTGCATACC
CGTGTTACTACCTTTACTTTCCCGAACCCGTTCCCGTTTGATCGTAACGG
TAACGCTGTTTACGAACTGTCCAACACTAACTGGAAATGCTTCTTCGAGC
GTCTGTCTTCCTCCCTGGACATCCAGGACTCTGAAGATGAAGAAGATGGT
TCTAACTCTCAGGCTTTCCGTTGTGTTCCGGGTACTGTTGTTCGTACTCT
GATGGAAGCCATCGCGAAGAGGCTCGACGCCTGCCAGGACCAGCTGCTCG
AGCTGTACGAGGAGAACAGCATTGACATCCATAAGCACATCATGCACTGG
AAGTGCATTCGCCTGGAGAGCGTGCTGTTGCACAAGGCCAAGCAGATGGG
CCTGTCCCACATAGGCCTTCAGGTGGTCCCCCCTCTGACCGTGTCAGAGA
CAAAGGGCCATAACGCAATCGAGATGCAGATGCACCTCGAGTCGCTGGCG
AAAACACAGTACGGCGTGGAGCCATGGACCCTGCAGGACACCTCGTACGA
AATGTGGCTGACCCCACCTAAGCGATGCTTCGCCAAACAGGGCAACACAG
TGGAGGTGAAGTTCGACGGCTGTGAGGATAACGTTATGGAGTATGTCGTG
TGGACGCACATCTATCTGCAGGACAACGACAGTTGGGTGAAGGTGACCAG
CTCCGTGGACGCGAAGGGCATCTACTATACCTGTGGGCAGTTTAAAACCT
ACTATGTGAACTTCAACAAAGAGGCCCAAAAGTATGGCTCCACCAACCAC
TGGGAGGTCTGCTATGGGAGCACGGTGATTTGCTCTCCCGCCAGCGTGTC
TAGCACTGTGCGCGAGGTGAGCATTGCCGAGCCGACCACGTACACCCCTG
CCCAGACGACCGCTCCGACCGTGTCTGCTTGTACTACCGAGGACGGCGTG
AGCGCTCCACCCAGGAAGCGTGCGAGGGGCCCAAGCACCAACAACACCCT
CTGTGTGGCGAACATTCGCAGCGTCGACAGTACCATCAATAACATCGTGA
CGGATAACTATAACAAGCACCAGAGGCGTAACAACTGTCACTCTGCCGCA
ACCCCCATCGTGCAGCTCCAGGGAGACAGCAATTGCCTTAAGTGCTTCCG
CTATCGCCTCAACGACAAGTACAAGCACCTCTTTGAGCTCGCCTCGTCGA
CGTGGCACTGGGCCTCACCCGAGGCACCTCACAAGAACGCCATCGTCACT
CTCACTTACTCCAGTGAGGAGCAGAGACAGCAGTTTCTGAACAGCGTGAA
GATCCCACCGACGATCCGTCATAAGGTCGGCTTCATGTCACTGCATCTCC
TGTGA
Amino acid sequence (Seq. ID No. 20)
MEAIAKRLDA CQEQLLELYE ENSTDLHKHV LHWKCMRHES
VLLYKAKQMG LSHIGMQVVP
PLKVSEAKGH NAIEMQMHLE SLLRTEYSME PWTLQETSYE
MWQTPPKRCF AKRGKTVEVK
FDGCANNTMD YVVWTDVYVQ DNDTWVKVHS MVDAKGIYYT
CGQFKTYYVN FVKEAEKYGS
TKHWEVCYGS TVICSPASVS STTQEVSIPE STTYTPAQTS
TLVSSSTKED AVQTPPRKRA
RGVQQSPCNA LCVAHIGPVD SGNHNLITNN HDQHQRRNNS
NSSATPIVQF QGESNCLKCF
RYRLNDRHRH LFDLISSTWH WASSKAPHKH AIVTVTYDSE
EQRQQFLDVV KIPPTISHKL
GFMSLHLLMA DDSGTENEGS GCTGWFMVEA IVQHPTGTQI
SDDEDEEVED SGYDMVDFID
DSNITHNSLE AQALFNRQEA DTHYATVQDL GGKYLGSPYV
SPINTIAEAV ESEISPRLDA
IKLTRQPKKV KRRLFQTREL TDSGYGYSEV EAGTGTQVEK
HGVPENGGDG QEKDTGRDIE
GEEHTEAEAP TNSVREHAGT AGILELLKCK DLRAALLGKF
KECFGLSFID LIRPFKSDKT
TCLDWVVAGF GIHHSISEAF QKLIEPLSLY AHIQWLTNAW
GMVLLVLLRF KVNKSRSTVA
RTLATLLNIP ENQMLIEPPK IQSGVAALYW FRTGISNAST
VIGEAPEWIT RQTVIEHGLA
DSQFKLTEMV QWAYDNDICE ESEIAFEYAQ RGDFDSNARA
FLNSNMQAKY VKDCATMCRH
YKHAEMRKMS IKQWIKHRGS KIEGTGNWKP IVQFLRHQNI
EFIPFLTKFK LWLHGTPKKN
CIAIVGPPDT DKSYFCMSLI SFLGGTVISH VNSSSHFWLQ
PLVDAKVALL DDATQPCWIY
MDTYMRNLLD GNPMSIDRKH KALTLIKCPP LLVTSNIDIT
KEDKYKYLHT RVTTFTFPNP
FPFDRNGNAV YELSNTNWKC FFERLSSSLD IQDSEDEEDG
SNSQAFRCVP GTVVRTLMEA
IAKRLDACQD QLLELYEENS IDIHKHIMHW KCIRLESVLL
HKAKQMGLSH IGLQVVPPLT
VSETKGHNAI EMQMHLESLA KTQYGVEPWT LQDTSYEMWL
TPPKRCFAKQ GNTVEVKFDG
CEDNVMEYVV WTHIYLQDND SWVKVTSSVD AKGIYYTCGQ
FKTYYVNFNK EAQKYGSTNH
WEVCYGSTVI CSPASVSSTV REVSIAEPTT YTPAQTTAPT
VSACTTEDGV SAPPRKRARG
PSTNNTLCVA NIRSVDSTIN NIVTDNYNKH QRRNNCHSAA
TPIVQLQGDS NCLKCFRYRL
NDKYKHLFEL ASSTWHWASP EAPHKNAIVT LTYSSEEQRQ
QFLNSVKIPP TIRHKVGFMS
LHLL

### 11. PLASMID: HPV118 (p7313ie 6be2.11e2.6be1)

Gene of Interest:

The gene for the polyprotein in construct HPV118 is a triple fusion protein comprised in order of 6be2, 11e2, 6be1 all codon optimised and mutated. The polyprotein gene was assembled by PCR from using 2 previous PCR fragments; 6be1 and 11/6be2. The size of the gene is 4.1 kb, producing a polyprotein of 170 kD, observed by PAGE and Western blot.

Cloning:

The polyprotein gene was digested with Bam HI+Not I restriction enzymes and ligated into p7313ie vector. Sequencing analysis of selected clones had indicated the ‘odd’ base change, but this was overcome by various fragment swapping. A resulting clone hpv118 #3 was found to have no errors.

Polyprotein sequence in HPV118 (Seq. ID No. 21)
ATGGAAGCTATTGCCAAGCGACTGGACGCCTGCCAGGAGCAGCTGCTGGA
GCTGTACGAGGAAAACAGCACAGACCTCCACAAGCACGTGCTGCACTGGA
AGTGCATGCGCCACGAGTCAGTGCTCCTGTACAAGGCCAAGCAGATGGGG
CTGTCCCACATCGGGATGCAGGTCGTGCCCCCGCTGAAGGTGAGCGAAGC
CAAGGGCCACAACGCTATCGAGATGCAGATGCACCTGGAGAGCCTGCTGC
GGACCGAATACAGCATGGAGCCCTGGACTCTCCAGGAGACGTCCTACGAA
ATGTGGCAGACTCCTCCGAAGCGCTGTTTCGCAAAGCGCGGCAAGACAGT
TGAGGTGAAATTCGATGGGTGCGCAAACAACACGATGGACTACGTGGTGT
GGACCGATGTCTACGTGCAGGACAATGACACCTGGGTGAAGGTACATAGT
ATGGTGGATGCCAAGGGCATCTATTACACCTGCGGGCAGTTCAAGACGTA
CTACGTCAACTTCGTCAAGGAAGCCGAAAAGTATGGTTCCACCAAGCACT
GGGAGGTGTGTTACGGGAGTACTGTGATCTGCAGCCCCGCCTCCGTGTCG
TCCACCACCCAGGAAGTGAGCATTCCGGAGAGCACCACATACACCCCGGC
CCAAACGAGCACGCTCGTCAGCAGCAGCACCAAGGAGGACGCCGTCCAGA
CGCCCCCCCGGAAGAGGGCCCGGGGGGTCCAGCAGTCTCCCTGCAATGCC
CTGTGCGTTGCTCACATCGGCCCTGTCGATTCTGGGAACCACAATCTCAT
CACGAACAACCACGACCAGCACCAAAGGCGCAACAACTCTAACAGCTCCG
CAACTCCAATAGTGCAGTTCCAGGGGGAGTCCAACTGCCTCAAGTGTTTC
CGCTACCGCCTCAACGACCGCCACCGCCACCTGTTCGACTTGATCAGTTC
CACGTGGCACTGGGCCAGCAGCAAGGCGCCCCACAAACACGCTATCGTGA
CGGTGACCTACGACTCCGAGGAGCAGAGGCAGCAGTTCCTGGACGTCGTG
AAGATTCCTCCGACAATCAGCCACAAGCTTGGCTTCATGTCCCTGCACCT
GCTGATGGAAGCCATCGCGAAGAGGCTCGACGCCTGCCAGGACCAGCTGC
TCGAGCTGTACGAGGAGAACAGCATTGACATCCATAAGCACATCATGCAC
TGGAAGTGCATTCGCCTGGAGAGCGTGCTGTTGCACAAGGCCAAGCAGAT
GGGCCTGTCCCACATAGGCCTTCAGGTGGTCCCCCCTCTGACCGTGTCAG
AGACAAAGGGCCATAACGCAATCGAGATGCAGATGCACCTCGAGTCGCTG
GCGAAAACACAGTACGGCGTGGAGCCATGGACCCTGCAGGACACCTCGTA
CGAAATGTGGCTGACCCCACCTAAGCGATGCTTCGCCAAACAGGGCAACA
CAGTGGAGGTGAAGTTCGACGGCTGTGAGGATAACGTTATGGAGTATGTC
GTGTGGACGCACATCTATCTGCAGGACAACGACAGTTGGGTGAAGGTGAC
CAGCTCCGTGGACGCGAAGGGCATCTACTATACCTGTGGGCAGTTTAAAA
CCTACTATGTGAACTTCAACAAAGAGGCCCAAAAGTATGGCTCCACCAAC
CACTGGGAGGTCTGCTATGGGAGCACGGTGATTTGCTCTCCCGCCAGCGT
GTCTAGCACTGTGCGCGAGGTGAGCATTGCCGAGCCGACCACGTACACCC
CTGCCCAGACGACCGCTCCGACCGTGTCTGCTTGTACTACCGAGGACGGC
GTGAGCGCTCCACCCAGGAAGCGTGCGAGGGGCCCAAGCACCAACAACAC
CCTCTGTGTGGCGAACATTCGCAGCGTCGACAGTACCATCAATAACATCG
TGACGGATAACTATAACAAGCACCAGAGGCGTAACAACTGTCACTCTGCC
GCAACCCCCATCGTGCAGCTCCAGGGAGACAGCAATTGCCTTAAGTGCTT
CCGCTATCGCCTCAACGACAAGTACAAGCACCTCTTTGAGCTCGCCTCGT
CGACGTGGCACTGGGCCTCACCCGAGGCACCTCACAAGAACGCCATCGTC
ACTCTCACTTACTCCAGTGAGGAGCAGAGACAGCAGTTTCTGAACAGCGT
GAAGATCCCACCGACGATCCGTCATAAGGTCGGCTTCATGTCACTGCATC
TCCTGATGGCAGACGATTCCGGTACTGAGAACGAAGGTTCTGGTTGTACC
GGTTGGTTCATGGTTGAAGCAATCGTTCAGCATCCGACTGGTACCCAGAT
CTCCGATGACGAAGACGAAGAAGTTGAAGATTCTGGTTACGACATGGTTG
ACTTCATCGATGACTCCAACATCACTCATAACTCTCTGGAAGCACAGGCT
CTGTTTAACCGCCAGGAAGCTGATACCCATTACGCTACTGTTCAGGACCT
GGGAGGCAAATATCTGGGCTCTCCGTACGTTTCCCCGATCAACACTATCG
CAGAAGCAGTTGAGTCTGAAATCTCCCCGCGCCTGGACGCTATCAAACTG
ACTCGTCAGCCGAAGAAGGTTAAACGTCGTCTGTTCCAGACTCGTGAACT
GACCGACTCCGGTTACGGTTATAGCGAAGTTGAGGCTGGCACCGGCACCC
AGGTTGAAAAACACGGTGTACCGGAAAACGGCGGCGACGGTCAGGAAAAG
GACACCGGCCGCGACATCGAGGGTGAGGAACACACCGAAGCTGAAGCTCC
GACTAACTCTGTTCGTGAACACGCAGGTACTGCGGGTATCCTGGAACTGC
TGAAATGCAAAGACCTGCGCGCGGCTCTGCTGGGCAAATTCAAAGAATGC
TTCGGCCTGTCTTTCATTGACCTGATCCGTCCGTTTAAGTCTGACAAAAC
TACCTGTCTGGACTGGGTTGTAGCAGGCTTCGGCATCCACCACTCTATCT
CTGAAGCATTCCAGAAACTGATCGAGCCGCTGTCTCTGTACGCGCACATC
CAGTGGCTGACTAACGCTTGGGGTATGGTTCTGCTGGTACTGCTGCGCTT
TAAAGTAAACAAATCTCGTTCCACTGTTGCTCGTACTCTGGCTACCCTGC
TGAACATCCCGGAGAACCAGATGCTGATCGAACCGCCGAAAATCCAGTCT
GGTGTAGCTGCACTGTACTGGTTTCGTACTGGCATCTCTAACGCTAGCAC
TGTTATCGGTGAAGCACCGGAATGGATCACTCGTCAGACCGTTATCGAAC
ACGGTCTGGCAGATTCTCAGTTCAAACTGACTGAAATGGTTCAGTGGGCA
TACGACAACGACATCTGCGAGGAATCTGAAATTGCGTTCGAATACGCTCA
GCGTGGCGACTTCGACTCCAACGCTCGTGCTTTCCTGAACAGCAACATGC
AGGCTAAATACGTAAAAGACTGCGCTACCATGTGCCGTCACTACAAACAC
GCGGAAATGCGTAAAATGTCTATCAAACAGTGGATCAAGCACCGCGGTTC
TAAAATCGAAGGTACCGGTAACTGGAAACCGATCGTTCAGTTCCTGCGCC
ATCAGAACATCGAATTCATCCCGTTCCTGACCAAATTCAAGCTGTGGCTG
CACGGTACCCCGAAAAAAAACTGCATCGCTATCGTAGGTCCACCGGACAC
TGACAAGTCTTACTTCTGTATGTCCCTGATCTCTTTCCTGGGCGGCACTG
TAATCTCTCACGTTAACTCTTCCTCCCATTTCTGGCTGCAGCCACTGGTA
GACGCGAAAGTAGCTCTGCTGGACGACGCGACCCAGCCGTGCTGGATCTA
CATGGATACTTACATGCGCAACCTGCTGGACGGTAACCCGATGTCTATCG
ACCGTAAACACAAAGCGCTGACTCTGATCAAGTGCCCGCCGCTGCTGGTA
ACTTCTAACATCGACATCACCAAGGAAGATAAATACAAGTACCTGCATAC
CCGTGTTACTACCTTTACTTTCCCGAACCCGTTCCCGTTTGATCGTAACG
GTAACGCTGTTTACGAACTGTCCAACACTAACTGGAAATGCTTCTTCGAG
CGTCTGTCTTCCTCCCTGGACATCCAGGACTCTGAAGATGAAGAAGATGG
TTCTAACTCTCAGGCTTTCCGTTGTGTTCCGGGTACTGTTGTTCGTACTC
TGTGA
Amino acid sequence (Seq. ID No. 22)
MEAIAKRLDA CQEQLLELYE ENSTDLHKHV LHWKCMRHES
VLLYKAKQMG LSHIGMQVVP PLKVSEAKGH NAIEMQMHLE
SLLRTEYSME PWTLQETSYE MWQTPPKRCF AKRGKTVEVK
FDGCANNTMD YVVWTDVYVQ DNDTWVKVHS MVDAKGIYYT
CGQFKTYYVN FVKEAEKYGS TKHWEVCYGS TVICSPASVS
STTQEVSIPE STTYTPAQTS TLVSSSTKED AVQTPPRKRA
RGVQQSPCNA LCVAHIGPVD SGNHNLITNN HDQHQRRNNS
NSSATPIVQF QGESNCLKCF RYRLNDRHRH LFDLISSTWH
WASSKAPHKH AIVTVTYDSE EQRQQFLDVV KIPPTISHKL
GFMSLHLLME AIAKRLDACQ DQLLELYEEN SIDIHKHIMH
WKCIRLESVL LHKAKQMGLS HIGLQVVPPL TVSETKGHNA
IEMQMHLESL AKTQYGVEPW TLQDTSYEMW LTPPKRCFAK
QGNTVEVKFD GCEDNVMEYV VWTHIYLQDN DSWVKVTSSV
DAKGIYYTCG QFKTYYVNFN KEAQKYGSTN HWEVCYGSTV
ICSPASVSST VREVSIAEPT TYTPAQTTAP TVSACTTEDG
VSAPPRKRAR GPSTNNTLCV ANIRSVDSTI NNIVTDNYNK
HQRRNNCHSA ATPIVQLQGD SNCLKCFRYR LNDKYKHLFE
LASSTWHWAS PEAPHKNAIV TLTYSSEEQR QQFLNSVKIP
PTIRHKVGFM SLHLLMADDS GTENEGSGCT GWFMVEAIVQ
HPTGTQISDD EDEEVEDSGY DMVDFIDDSN ITHNSLEAQA
LFNRQEADTH YATVQDLGGK YLGSPYVSPI NTIAEAVESE
ISPRLDAIKL TRQPKKVKRR LFQTRELTDS GYGYSEVEAG
TGTQVEKHGV PENGGDGQEK DTGRDIEGEE HTEAEAPTNS
VREHAGTAGI LELLKCKDLR AALLGKFKEC FGLSFIDLIR
PFKSDKTTCL DWVVAGFGIH HSISEAFQKL IEPLSLYAHI
QWLTNAWGMV LLVLLREKVN KSRSTVARTL ATLLNIPENQ
MLIEPPKIQS GVAALYWFRT GISNASTVIG EAPEWITRQT
VIEHGLADSQ FKLTEMVQWA YDNDICEESE IAFEYAQRGD
FDSNARAFLN SNMQAKYVKD CATMCRHYKH AEMRKMSIKQ
WIKHRGSKIE GTGNWKPIVQ FLRHQNIEFI PFLTKFKLWL
HGTPKKNCIA IVGPPDTDKS YFCMSLISFL GGTVISHVNS
SSHFWLQPLV DAKVALLDDA TQPCWIYMDT YMRNLLDGNP
MSIDRKHKAL TLIKCPPLLV TSNIDITKED KYKYLHTRVT
TFTFPNPFPF DRNGNAVYEL SNTNWKCFFE RLSSSLDIQD
SEDEEDGSNS QAFRCVPGTV VRTL

The ColE1 cer sequence was obtained from a subclone from plasmid pDAH212 from David Hodgeson (Warwick University) and amplified by PCR using primers to place EcoRI restriction sites at the ends of the sequence. The cer sequence was then inserted into the EcoRI site of p7313-PL to produce plasmid p7313-PLc. The sequence of the amplified cer was verified against the Genbank entry M11411.

### Example 2

### Expression in Mammalian 293T Cells

Mammalian 293T cells were grown at log phase at a final concentration of 2×10⁵ cells per 6 well Corning Costar™ (Corning Science Products, 10 The ValleyCentre, Gordon Road, High Wycombe, Bucks, UK) tissue culture plate overnight at 37° C. in 5% CO₂. The following transfection mix was prepared and complexed for 25 minutes:

| | | |
|---|---|---|
| DNA of Interest | 2 | μg |
| | 2 | μg |
| Made up with sterile double distilled water | 16 | μl |
| OPTI-mem ™ (Gibco BRL, Paisley, Scotland) | 8 | μl |
| Lipofectamine ™ (GibcoBRL) | 6 | μl. |

Each cell monolayer in a well was washed carefully twice with OPTI-mem™. 800 μl of OPTI-mem™ was added to each well. 200 μl of OPTI-mem™ was added to each transfection mix, mixed and added gently to a cell monolayer. The plate was incubated for 5 hours at 37° C. in 5% CO₂ after which the transfection mix and OPTI-mem™ were discarded. The cell monolayers were washed gently with cell growth medium twice and finally transfected cells were incubated for 24 hours in Dulbecco's Modified Eagle Medium containing 10% foetal calf serum and 29.2 mg/ml of L-glutamine at 37° C. in 5% CO₂. The cells were scraped off into microtubes, washed twice with PBS, spun down and the cell pellet was resuspended in SDS Page Laemmli dye. The cell pellets were boiled and loaded onto a 10% SDS Page gel, electrophoresed in 1×Tris Glycine SDS buffer. After electrophoresis, the gel was blotted onto Nitrocellulose membrane (Amersham) and Western Blotted. The nitrocellulose membrane was blocked with 5% Marvel™ (Premier Beverages, Knighton, Adbaston, Stafford, UK) in PBS for 30 min at room temperature and washed twice with PBS and 0.1% Tween 20. A polyclonal antibody raised against the C terminal protein sequence of HPV6bE1 (protein sequence: CSSSLDIQDSEDEEDGSNSQAFR Seq. ID No. 23) in rabbits, was diluted in 5% Marvel™ in PBS and added to the nitrocellulose membrane. This was incubated at room temperature for 1 hour with gentle agitation. A polyclonal antibody against HPV11E1 was also used to check cross reactivity. The diluted antibody was removed and the membrane washed three times with PBS and 0.1% Tween 20. A secondary conjugate, Swine anti-rabbit horseradish peroxidase (HRP) (DAKO), was diluted 1:20000 in PBS and 0.1% Tween 20. This was added to the washed membrane and incubated with gentle agitation at room temperature for 1 hour. The membrane was then washed thoroughly with PBS and 0.1% Tween 20. A Chemiluminescent HRP kit (Amersham) was used to detect the transferred proteins on the membrane.

Results:

The results (FIG. 13) show a correct protein size expressed by each of HPV 116, 117, 118 containing the codon optimised HPV polyproteins.

HEK293T cells were transfected with ˜0.5 ug DNA of the respective constructs and the cells harvested 24 hrs later. These samples were then analysed by first polyacrylamide electrophoresis and then Western blotting. Two peptide antibodies were used to detect for polyprotein expression (˜180 kd); Anti-6bE1 (no. 1097) and anti-6bE2 (no. 1101).

### Example 3

### E1 Antigen Inactivation and Experimental Confirmation

The HPV E1 protein is a well conserved nuclear protein with non-specific DNA binding, ATPase and helicase activities. E1 also binds to host cellular DNA polymerase-α primase and, to the HPV E2 protein which then ‘recruits’ E1 into the pre-initiation viral DNA replication complex. The primary role of E1 is to initiate virus specific DNA replication in infected cells.

The DNA replication functions of E1 (and E2) are relatively non-specific and many studies have now shown that the E1 and E2 proteins from one genotype can drive the origin specific DNA replication of a plasmid carrying the replication origin sequence from a different genotype. Studies have also shown that the introduction of highly expressed E1 and E2 into cells already harbouring low copy number HPV plasmid can result in a significant amplification of that plasmid. This promiscuity carries with it a small potential safety risk which the project sought to eliminate. Consequently, mutations in E1 (and E2) which inactivate their replication potential were sought.

The E1 mutation G482D occurs in a highly conserved ATP binding consensus sequence and E1 protein carrying this mutation has been shown to have multiple functional deficits. Other mutations, towards the N-terminus of the protein (K83G, R84G) have been shown to abrogate nuclear localisation of E1. Failure to locate to the nuclear compartment would also serve to separate E1 from host replication proteins and viral DNA, providing an additional level of incapacity and safety. These mutations (G428D, K83G, R84G) were selected and incorporated into E1 as part of the HPV DNA immunotherapeutic E1 vector.

An in vitro HPV DNA replication assay was used to confirm disablement of the DNA replication functions of E1 (as a corollary the mutational inactivation of the replication enhancing activity of E2 could also be confirmed in this same assay). Briefly, both E1 and E2 co-operatively activate the HPV origin of replication and the E1 and E2 proteins from HPV 6b ware known to activate and drive de novo DNA replication from the HPV-11 origin. Plasmids encoding our codon-optimised E1 and E2 sequences were co-transfected into 293 cells with a plasmid carrying the HPV-11 origin of replication (ori plasmid). E1 and E2 dependent replication of the input ori plasmid is measured by harvesting DNA from cells 48 hours after co-transfection (Hirt lysis). Extracted DNA is restriction enzyme digested first with Hind III and then Dpn I which digests unmethylated unreplicated DNA. DNA's are then southern blotted and hybridised with ori plasmid DNA as probe. Bands with a size equivalent to ori plasmid after DpnI digestion are markers for de novo in vitro replicated plasmid DNA.

Wild type E1 and E2 (HPV 119+HPV 120) show a strong band indicative of replicated input plamsid DNA. Each of the three lead constructs are negative, (HPV116, HPV117 and HPV118) showing results; no replication.

Conclusion:

The lead constructs HPV 116, HPV 117 and HPV 118 have no DNA replication activity.

### Example 4

The E2 protein of papillomaviruses is a site-specific DNA binding nuclear protein functioning as the primary replication origin recognition protein and assists in the assembly of the pre-initiation DNA replication complex. Full length E2 protein can also act as either a repressor or activator of viral transcription depending upon the position (relative to other transcription factor sites), and the affinity of the protein for its cognate binding site. E2 is also known to influence the transcription of several host cellular promoters. The mutational inactivation of E2 has been studied extensively and one point mutation in particular Lys 111→Ala (K111A) has been shown to inactivate both the transcriptional and replication functions of E2. This mutation may also have the addition benefit of preventing nuclear translocation of the protein. This mutation (K111A) was incorporated into each E2 antigen as part of the HPV DNA immunotherapeutic.

We set out to confirm the incapacity of K111A mutated E2 and each polyprotein construct in an in vitro CAT transcriptional reporter assay. We used two positive controls (sources of active E2 protein). These were a construct expressing unmutated (active) HPV-11E2 protein, and a second vector expressing BPV E2 protein, a strong transcriptional transactivator. These data are shown in FIG. 14.

Conclusion:

These data show that protein expressed from the native (unmutated) HPV 6b E2 vector is transcriptionally active, whilst mutated (K111A) E2 is inactive, as are each of the polyprotein vectors HPV 116, 117 and 118.

### Example 5

### Expression of and Comparison with Individual Gene Constructs HPV 116, HPV 117 and HPV 118

Gene expression studies comparing the leads constructs HPV 116, HPV 117 and HPV 118 failed to identify any clear differences in in vitro gene expression. In addition, expression of the polyprotein was equivalent to expression of the individual (unfused) antigen in a single plasmid (HPV 110).

Equally important, the introduction of the point mutations did not impact on gene expression (HPV 108 and HPV 110).

### Example 6

### In Vivo Immunogenicity Studies in Mice

In order to compare the immunogenicity of the three different constructs HPV 116, HPV 117 and HPV 118 in vivo, mice were immunised using PMID.

Each immunisation comprised two shots of 0.5 □g DNA fired into the shaved abdomen of Balb/c (H-2K^{d}) or C57 BL6 (H-2K^{b}) mice. Animals were primed with 1 □g DNA, boosted 21 days later with an equivalent dose and culled 5-7 days post boost. Sera and spleens were taken for analysis of the humoral and cellular immune response generated following PMID.

Humoral Assays

Antibodies raised in PMID immunised mice were evaluated using standard ELISA methods and recombinant E1 and E2 protein as capture antigen. Antibody responses could not be reliably detected except after extended immunisation schedules in E2 immunised mice. We did not confirm detection of antibody to the E1 antigen in mice. These weak/undetectable antibody responses are in keeping with the published literature.

Cellular Assays

ELISPOT assays were used to study cellular immune responses in mice. This technique is suitable for assessing the frequency of cells within a culture of known density that are capable of secreting cytokines specifically in response to antigen presented in the context of syngeneic MHC molecules.

Briefly, a single cell suspension of splenocytes isolated from immunised animals is added to specialised microtitre plates coated with anti-cytokine capture antibody and incubated overnight in the presence of antigen presented by suitable target cells. Cytokine is captured by antibody bound to the plate in the area directly around the cell and this remains bound when cells are lysed and washed away. Detection is achieved by use of a biotinylated secondary anti-cytokine antibody and a streptavidin alkaline phosphatase conjugate. The action of this enzyme on a chromophoric substrate allows visualisation of the frequency of cytokine producing cells.

Vaccinia ELISPOT Assays and Data

Due to the absence of defined murine T cell epitopes, antigen was provided in the form of recombinant vaccinia viruses engineered to express target antigens. Such viruses were used to infect appropriate target cells for the presentation of antigen to effector cells in ELISPOT assays.

Responses to HPV 6bE1 were detected following PMID of the three candidate constructs to C57BL/6 mice. The results of 2 separate experiments were analysed statistically. The results of a representative experiment are shown in the FIGS. 15 and 16.

Illustrative immunogenicity data using lead constructs and PMID in mice:

CTL Assays and Data

Activated CD8+ T cells are able to lyse cells in response to specific peptide presented in the context of syngeneic MHC I molecules. This function can be determined by Eu3+ release bioassay, a non-radioactive modification of the traditional chromium release assay.

Use of this assay for these purposes required the identification of a CD8+ T cell epitope derived from the primary sequence of the HPV 6bE1 protein. This was achieved by screening a peptide library consisting of 15-mers overlapping by 11 using cytokine ELISPOT. Responding populations were identified as CD4+ or CD8+ T cells by standard flow techniques.

The basis of this technique involves lysis of Eu3+ labeled target cells pulsed with cognate peptide. During the course of a two hour incubation, Eu3+ is released into the culture supernatant upon lysis of target cells by cytolytic T cells. This is detected by time-resolved fluorimetry. Specific lysis is expressed as a percentage of the total amount of lysis detected when target cells are lysed by chemical means.

Assessment of Cellular Immunology Data

The immunologic evaluation of HPV 116, HPV 117 and HPV 118, comprised repeat PMID immunisation studies in mice with Vaccinia ELISPOT and CTL assay analysis as immunologic outputs. All candidates raised a strong immune response to each antigen.

Collectively, the vaccinia ELISPOT data show that responses to E1 are not compromised by mutation or by fusion to the E2 antigen components. When comparing E1 responses between HPV-108 (single 6b E1 construct), HPV 116, HPV 117 and HPV 118 the responses are not statistically different. Vaccinia ELISPOT data do however reveal a difference in responses to the HPV-1 E2 antigen component. E2 antigen specific responses are significantly greater in mice immunised with HPV 118 than in mice immunised with HPV 116 or HPV 117. On this basis alone HPV 118 appears to be a superior immunogen than HPV 116 or HPV 117.

The analysis of E1 antigen specific CTL lysis also revealed a trend in potency. The percentage specific lysis was higher using T-cells form HPV 118 immunised mice than with either of HPV 116 or HPV 117. This observation is reproducible.

Taken together, and on the basis of both vaccinia ELISOT and CTL lysis data, HPV 118 is the stronger immunogen.

Conclusion.

On purely immunological criteria construct HPV 118 is the most immunogenic of the polyproteins.

### Example 7

### PMID Delivery of Codon-Optimised COPV E1/E2 Fusion Protein is More Effective in Protecting Against Canine Oral Papillomavirus Disease than Either Codon-Optimised E1 or Codon-Optimised E2 Alone

Introduction

The canine oral papillomavirus (COPV) animal model is a good mimic of mucosal human papillomavirus disease. The features of disease caused in dogs by COPV are very similar to that which occurs in humans (Nicholls et al Virology 2001, 283(1) 31-39). Importantly it is a mucosal papillomavirus disease model. The COPV virus infects the canine mucosal epithelia and, after a lag period of a few weeks warts appear which then regress spontaneously after an additional period of some weeks. The COPV virus encodes homologues of each of the human papillomavirus genes (E1, E2, E4, E6, E7, L1 and L2).

The dog COPV mucosal disease model has previously been used as a key model in developing the rationale for human virus-like-particle (VLP) papillomavirus vaccines (Ghim et al, Vaccines 1995 25, 375-379, Suzich et al, PNAS 1995, 92 11553-11557). Human papillomavirus VLP vaccines are now in development, and early stage clinical trials have recently been completed in humans.

We show that plasmid DNA encoding a codon-optimised fusion of E1 and E2 genes when administered by PMID reduces disease burden more effectively than either than either a plasmid encoding codon-optimised E1 or codon-optimise E2 alone.

Methods

Construction of the Codon-Optimised E2/E 1 Fusion Vector

A synthetic gene encoding a codon-optimised COPV E2 sequence was generated using methods described previously. This was fused to the synthetic codon-optimised COPV E1 gene recovered from clone pCOPVE1 c/o and inserted into vector WRG7077 to generate a new clone which was designated pCOPVE2/E1 c/o. This clone expresses a polyprotein comprising a fusion of COPV E2 (N terminal) and COPV E1 (C terminal). The polyprotein is of the expected size as determined by western blotting.

Immunisation of Beagle Dogs with pCOPVE1 c/o, pCOPVE2 c/o, and pCOPVE2/E1 c/o

Beagle dogs were immunised by PMID with each of three purified plasmids pCOPVE1 c/o, pCOPVE2 c/o and, pCOPV E2/E1 c/o. Animal were immunised at 12 cutaneous sites, 6 non-overlapping sites on each side of the abdominal midline. All vaccinations were performed under general anesthesia. There were five animals in each group. Six weeks after the first vaccination, a boosting vaccination was undertaken in an identical manner, using the same procedure.

Immunised animals were challenged with infectious COPV virus 2 weeks after the final boosting immunisation. The mucosa of the upper lip of each animal was lightly scarified. 10 μl of purified COPV virus preparation was applied to each of ten sites (five on each side of the upper lip) and allowed to absorb for a few minutes. The isolation and purification of infectious COPV virus has been described (Virology 1999, 265 (2) 365-374).

After challenge with COPV virus the sites of mucosal challenge were examined weekly. The time (after challenge) of wart (papilloma) appearance, and wart size (mm) was measured.

In animals immunised with pCOPVE1 c/o papillomas developed at the mucosal challenge sites beginning at week 7 after challenge. Papillomas continued to grow in size reaching a mean size of >3.5 mm by week 11. In animals immunised with pCOPV E2 c/o papilloma's first appeared at week 8 but and the mean papilloma size reached 1.5 mm at week 11. In animals immunised with pCOPVE2/E1 c/o whilst the first signs of disease are co-incident with that of the other groups the overall disease burden is significantly reduced. One animal (of five) in the pCOPVE2/E1 c/o group was fully protected from disease development whilst all other animals in the group developed only very small papilloma's which regressed in a short period (1-2 weeks).

Plasmid DNA encoding a fusion of COPV E1 and COPV E2 are more effective than either of COPV E1 or COPV E2 in preventing disease development in this animal model of papillomavirus infection. (FIG. 18)

1. A polynucleotide sequence encoding a Human Papillomavirus (HPV) polypeptide having epitopes from at least three Early antigens or fragments thereof from at least two different HPV strains and wherein the polynucleotide has a codon usage coefficient for human genes of greater than 0.4 and less than 1.0

2. A polynucleotide as claimed in claim 1 wherein at least one antigen is from HPV E1 or fragment thereof.

3. A polynucleotide as claimed in claim 2 wherein at least one antigen is from HPV E2.

4. A polynucleotide sequence according to claim 1 which is a DNA sequence.

5. A polynucleotide sequence according to claim 1 which encodes a HPV polypeptide of a HPV type or sub-type associated with cervical cancer, benign cutaneous warts or genital warts.

6. A polynucleotide sequence according to claim 1 which encodes a HPV polypeptide of one of types 1-4, 6, 7, 10, 11, 16, 18, 26-29, 31, 33, 35, 39, 49, 51, 52, 56, 58, 59 and 68.

7. A polynucleotide sequence according to claim 6 which encodes a HPV polypeptide of an HPV type or sub-type which is associated with cervical cancer or genital warts.

8. A polynucleotide sequence according to claim 4 which encodes a HPV polypeptide of one of types 6, 11, 16, 18, 33 or 45.

9. A polynucleotide sequence according to claim 5 which encodes a HPV polypeptide of a HPV type or sub-type selected from HPV 11, 6a or 6b.

10. A polynucleotide sequence according to claim 1 in which encodes a mutated HPV polypeptide having reduced biological function.

11. A polynucleotide sequence according to claim 1 which encodes a mutated HPV polypeptide comprising one or more point mutations by which one or more of the polypeptide's natural biological functions is inactivated.

12. A polynucleotide sequence according to claim 1 comprising an epitope from E1 antigen of HPV 6b an epitope from HPV 6b E2, and an epitope from HPV 11 E2.

13. A polynucleotide sequence according to claim 1 having a codon usage coefficient for human genes of greater than 0.5 but less than 1.

14. An expression vector comprising a polynucleotide sequence according to claim 1 operably linked to a control sequence which is capable of providing for the expression of the polynucleotide sequence by a host cell.

15. An expression vector according to claim 14 which is p7313PLc.

16. A pharmaceutical composition comprising a polynucleotide sequence according to claim 1.

17. A pharmaceutical composition comprising a vector according to claim 14.

18. A pharmaceutical composition according to claim 16 comprising a plurality, gold particles, coated with DNA.

19. A pharmaceutical composition according to claim 16 further comprising an adjuvant.

20. A pharmaceutical composition according to claim 19 in which the adjuvant is encoded as a fusion with the HPV polypeptide encoded by the polynucleotide.

21. A method of treating or preventing HPV infections or any symptoms or diseases associated therewith, comprising administering an effective amount of a polynucleotide according to claim 1, a vector according to claim 14 or a pharmaceutical composition according to claim 16.

22. A method of treating or preventing HPV infections or any symptoms or diseases associated therewith, comprising administering a pharmaceutical composition according to claim 16 in a prime-boost dosage regime with a recombinant viral vector or non-viral based system comprising a polynucleotide according to claim 1.

23. The use of a composition according to claim 18 in the treatment or prophylaxis of an HPV infection.

24. The use of a polynucleotide according to claim 1, a vector according to claim 14 or a pharmaceutical composition according claim 16 in the treatment or prophylaxis of cutaneous (skin) warts, genital warts, atypical squamous cells of undetermined significance (ASCUS), cervical dysplasia, cervical intraepithelial neoplasia (CIN) or cervical cancer.

25. A method of treating or preventing HPV infections or any symptoms or diseases associated therewith, comprising administering an effective amount of a polynucleotide according to claim 1, a vector according to claim 14 or a pharmaceutical composition according to claim 16.

26. A method of treating or preventing HPV infections or any symptoms or diseases associated therewith, comprising administering a pharmaceutical composition according to in a prime-boost dosage regime with a recombinant viral vector or non-viral based system comprising a polynucleotide according to claim 1.
